SEQUENCE LISTING (8 sequences)

SEQ ID NO: 1 - protein, 214 aa, synthetic construct
  REGION 1..214: note = hum13B8-b light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, synthetic construct
  REGION 1..446: note = hum13B8-b heavy chain
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, synthetic construct
  REGION 1..10: note = HC CDR1
GYIFITYWMT

SEQ ID NO: 4 - protein, 16 aa, synthetic construct
  REGION 1..16: note = HC CDR2
QIFPASGSADYNEKFE

SEQ ID NO: 5 - protein, 7 aa, synthetic construct
  REGION 1..7: note = HC CDR3
GGGGFAY

SEQ ID NO: 6 - protein, 11 aa, synthetic construct
  REGION 1..11: note = LC CDR1
RTSENIYSYLA

SEQ ID NO: 7 - protein, 7 aa, synthetic construct
  REGION 1..7: note = LC CDR2
NAKTLAE

SEQ ID NO: 8 - protein, 9 aa, synthetic construct
  REGION 1..9: note = LC CDR3
QHHYGIPFT

(19) United States
(12) Patent Application Publication
(10) Pub. No.: US 2023/0303678 A1
(43) Pub. Date: Sep. 28, 2023

(54) METHODS FOR TREATMENT OF SUBJECTS WITH PLAQUE PSORIASIS OF THE SCALP

(71) Applicant: SUN PHARMACEUTICAL INDUSTRIES LIMITED, Mumbai (IN)

(72) Inventors: Judilyn FUENTES-DUCULAN, Scarsdale, NY (US); Tushar NISHANDAR, (US); Rocco BALLERINI, Doylestown, PA (US); Kristine NOGRALES, Green Brook, NJ (US); Siu-Long YAO, Washington Crossing, PA (US)

(73) Assignee: SUN PHARMACEUTICAL INDUSTRIES LIMITED, Mumbai (IN)

(21) Appl. No.: 18/158,767

(22) Filed: Jan. 24, 2023

(30) Foreign Application Priority Data
Jan. 25, 2022 (IN) 202221004128

(51) Int. Cl. C07K 16/24; A61P 17/06

(52) U.S. Cl. CPC C07K 16/244; A61P 17/06; A61K 2039/545

(57) ABSTRACT

The disclosure relates to an anti-IL-23p19 antibody hum13B8-b or antigen binding fragment thereof and its use in the treatment of plaque psoriasis of the scalp.

### CROSS REFERENCE TO RELATED APPLICATIONS

This application claims the benefit of Indian Application No. 202221004128, filed on Jan. 25, 2022, and which is incorporated by reference herein in its entirety.

### REFERENCE TO AN ELECTRONIC SEQUENCE LISTING

The instant application contains a Sequence Listing that has been submitted electronically and is hereby incorporated by reference in its entirety. The Sequence Listing was created on Jan. 23, 2023, is named “21-0347-WO_Sequence-Listing.xml” and is 11 kilobytes in size.

### FIELD OF THE DISCLOSURE

This disclosure relates to an anti-IL-23p19 antibody hum13B8-b or antigen binding fragment thereof and its use in the treatment of plaque psoriasis of the scalp. In some embodiments, the disclosure relates to a method of treating plaque psoriasis of the scalp wherein the treatment results in significant improvement from the patient's baseline score for the Investigator Global Assessment (IGA) mod 2011 Rating Scale (Scalp) score, IGA (scalp only) score, Scalp Itch Numeric Rating Scale (NRS) score, Psoriasis Scalp Severity Index (PSSI) score, Psoriasis Area and Severity Index (PASI) score, Physician Global Assessment of Skin (PGA-S) (whole body) score, and/or IGA mod 2011 (whole body) score. In some embodiments, the disclosure relates to a method of determining the efficacy of an anti-IL-23p19 antibody for the treatment of plaque psoriasis of the scalp.

### BACKGROUND

Psoriasis is a chronic, immune-mediated inflammatory disease characterized by the hyper-proliferation of keratinocytes and skin-infiltrating T-lymphocytes that overexpress pro-inflammatory mediators. The disease has a lifelong remitting and relapsing course with varying factors that trigger exacerbations in susceptible individuals. Psoriasis affects approximately 1% to 2% of people worldwide, with plaque psoriasis being the most common form that affects 80% to 90% of patients.

Of those affected by psoriasis, up to 80% will have involvement of the scalp. Scalp psoriasis may occur in isolation or in conjunction with other forms of psoriasis and is characterized by red, thickened plaques with silver-white scale, either contained within the hairline, or extending onto the forehead, ears, and posterior neck. Psoriasis of scalp can be a cause of great physical and social distress, with up to 97% of affected individuals reporting that the condition interferes in their daily life. In many cases, it is associated with intense pruritus, scale is commonly shed as dandruff creating significant social embarrassment for affected individuals and can result in alopecia. In a recent multinational survey, 43% of respondents to a telephone survey identified itch as the most bothersome symptom of their psoriasis (Crowley et al., JAMA Dermatol. 151(1): 87-94 (2015)).

Biological therapies are indicated for the treatment of subjects with moderate to severe chronic plaque psoriasis who are candidates for phototherapy or systemic therapy. Currently approved biological treatments include tumor necrosis factor (TNF) antagonist agents, a p40 (interleukin [IL]-12 and IL-23) antagonist, p19 (IL-23) antagonists, and IL-17 antagonists. Despite the availability of treatment options for psoriasis, scalp psoriasis lesions remain difficult to treat. The use of topical therapy, typically the first-line of treatment, is often challenging because of the inaccessibility of scalp lesions due to the presence of hair (Crowley, 2015 and Blakely et al., Psoriasis (Auckl.) 6: 33-40 (2016)). Patient satisfaction and compliance with topical therapy is known to be low (Devaux et al., J. Eur. Acad. Dermatol. Venereol. 26 Suppl 3: 61-67 (2012)). Thus, there remains an unmet need for effective therapeutic options for scalp psoriasis.

### SUMMARY

Provided herein is a method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

Also provided herein is a method of treating plaque psoriasis of the scalp comprising administering a therapeutically effective amount of an anti-IL-23p19 antibody hum13B8-b to a patient in need thereof, wherein administration of hum13B8-b results in an Investigator Global Assessment (IGA) mod 2011 rating scale (Scalp) score of “clear” or “almost clear” with at least a 2-point reduction from the patient's baseline score; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

Further provided herein is a method of treating plaque psoriasis of the scalp comprising administering a therapeutically effective amount of an anti-IL-23p19 antibody hum13B8-b to a patient in need thereof, wherein administration of hum13B8-b results in a significant improvement from the patient's baseline score in the Investigator Global Assessment (IGA) mod 2011 rating scale (Scalp) score; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

Further provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an Investigator Global Assessment (IGA) mod 2011 rating scale (Scalp) score of “clear” or “almost clear” with at least a 2-point reduction from the patient's baseline score at week 12, or week 16, or week 52 indicates the efficacy of the anti-IL-23p19 antibody.

Further provided herein is a method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein administration of hum13B8-b results in an Investigator Global Assessment (IGA) mod 2011 rating scale (Scalp) score of “clear” or “almost clear” with at least a 2-point reduction from the patient's baseline score.

Further provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an Investigator Global Assessment (IGA) mod 2011 rating scale (Scalp) score of “clear” or “almost clear” with at least a 2-point reduction from the patient's baseline score at week 52 indicates the efficacy of the anti-IL-23p19 antibody.

Further provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a 4-point reduction in Scalp Itch NRS score from the patient's baseline score at week 16 indicates the efficacy of the anti-IL-23p19 antibody.

Further provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient week 0, a second dose of hum13B8-b is subcutaneously administered to the patient at 4 weeks, and a subsequent dose hum13B8-b is subcutaneously administered to the patient every 4 to 12 weeks thereafter; wherein the hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a PGA-S score (whole body) of “clear” or “almost clear” with at least a 2-point reduction from the patient's baseline score at week 16 indicates the efficacy of the anti-IL-23p19 antibody.

Further provided herein is use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein a first dose of the medicament is subcutaneously administered to the patient on week 0, a second dose of the medicament is subcutaneously administered to the patient at 4 weeks, and a subsequent dose of the medicament is subcutaneously administered to the patient every 4 to 12 weeks thereafter.

Further provided herein is use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in an Investigator Global Assessment (IGA) mod 2011 rating scale (Scalp) score of “clear” or “almost clear” with at least a 2-point reduction from the patient's baseline score.

Further provided herein is use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in a significant improvement from the patient's baseline score in an Investigator Global Assessment (IGA) mod 2011 rating scale (Scalp) score.

Further provided herein is use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is subcutaneously administered to the patient on week 0, a second dose of the medicament is subcutaneously administered to the patient at 4 weeks, and a subsequent dose of the medicament is subcutaneously administered to the patient every 4 to 12 weeks thereafter, and wherein administration of the medicament results in an Investigator Global Assessment (IGA) mod 2011 rating scale (Scalp) score of “clear” or “almost clear” with at least a 2-point reduction from the patient's baseline score.

Further provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter.

Further provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in an Investigator Global Assessment (IGA) mod 2011 rating scale (Scalp) score of “clear” or “almost clear” with at least a 2-point reduction from the patient's baseline score.

Further provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in a significant improvement from the patient's baseline score in an Investigator Global Assessment (IGA) mod 2011 rating scale (Scalp) score.

Further provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter, and wherein administration of the pharmaceutical composition results in an Investigator Global Assessment (IGA) mod 2011 rating scale (Scalp) score of “clear” or “almost clear” with at least a 2-point reduction from the patient's baseline score.

Further provided herein is the use of an anti-IL-23p19 antibody hum13B8-b for the manufacture of a medicament for treating psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Further provided herein is a the use according to an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) score for the scalp of “clear” or “almost clear” with at least a 2-point reduction from Baseline at Week 16.

Further provided herein is the use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) score for the scalp of “clear” or “almost clear” with at least a 2-point reduction from Baseline at Week 12.

Further provided herein is the use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) Scalp Response Rate of 0 (clear) or 1 (almost clear) at Week 12.

Further provided herein is the use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving Psoriasis Scalp Severity Index (PSSI) 90 at Week 16.

Further provided herein is the use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving Psoriasis Scalp Severity Index (PSSI) 90 at Week 12.

Further provided herein is a pharmaceutical composition of an anti-IL-23p19 antibody hum13B8-b for the treatment of psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Further provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) score for the scalp of “clear” or “almost clear” with at least a 2-point reduction from Baseline at Week 16.

Further provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) score for the scalp of “clear” or “almost clear” with at least a 2-point reduction from Baseline at Week 12.

Further provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) Scalp Response Rate of 0 (clear) or 1 (almost clear) at Week 12.

Further provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the antibody results in the patient achieving Psoriasis Scalp Severity Index (PSSI) 90 at Week 16.

Further provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving Psoriasis Scalp Severity Index (PSSI) 90 at Week 12.

Further provided herein is a method of treating psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Further provided herein is a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) score for the scalp of “clear” or “almost clear” with at least a 2-point reduction from Baseline at Week 16.

Further provided herein is a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) score for the scalp of “clear” or “almost clear” with at least a 2-point reduction from Baseline at Week 12.

Further provided herein is a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) Scalp Response Rate of 0 (clear) or 1 (almost clear) at Week 12.

Further provided herein is a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving Psoriasis Scalp Severity Index (PSSI) 90 at Week 16.

Further provided herein is a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving Psoriasis Scalp Severity Index (PSSI) 90 at Week 12.

Further provided herein is a pharmaceutical composition of an anti-IL-23p19 antibody hum13B8-b for the manufacture of a medicament for treating psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

### BRIEF DESCRIPTIONS OF THE DRAWINGS

FIG. 1 is a schematic showing the study design. Abbreviations: n=number of subjects in each arm; R=randomization; SC=subcutaneous.

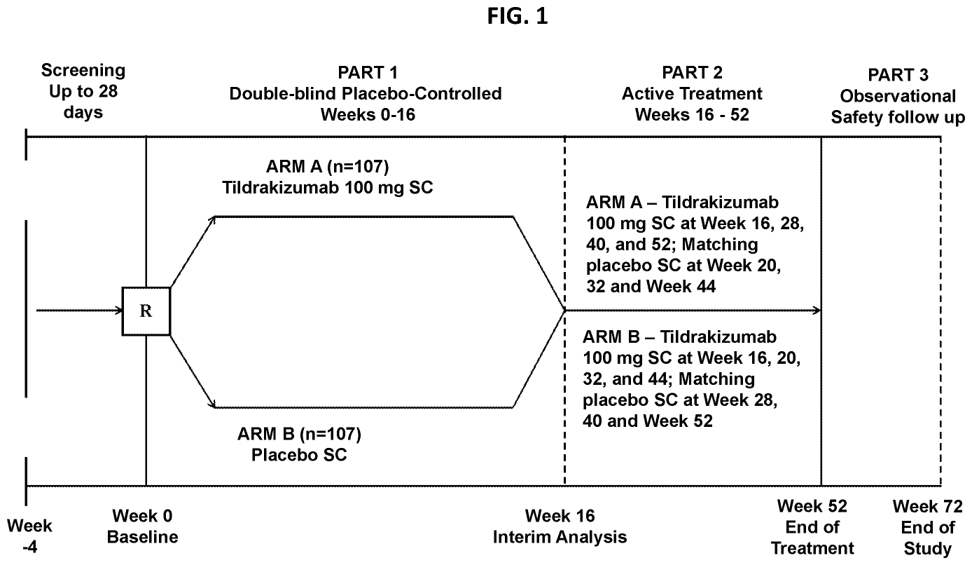

FIG. 2 is the Investigator Global Assessment (IGA) mod 2011 Rating Scale (Scalp).

FIG. 3 is the Investigator Global Assessment (IGA) (scalp only).

FIG. 4 is the Scalp Itch Numeric Rating Scale (NRS).

FIG. 5 is the Psoriasis Scalp Severity Index (PSSI).

FIG. 6 is the Investigator Global Assessment (IGA) mod 2011 Rating Scale (Whole Body).

FIG. 7 is the Physician Global Assessment of Skin (Whole Body).

FIG. 8 is the Psoriasis Area and Severity Index (PASI).

FIG. 9 is the Dermatology Life Quality Index (DLQI).

### DETAILED DESCRIPTION

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

It is noted that terms like “preferably,” “commonly,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

For the purposes of describing and defining the present disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Unless expressly specified otherwise, the term “comprising” is used in the context of the present disclosure to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

As utilized in accordance with the present disclosure, unless otherwise indicated, all technical and scientific terms shall be understood to have the same meaning as commonly understood by one of ordinary skill in the art.

The present disclosure relates to an anti-IL-23p19 antibody hum13B8-b or antigen binding fragment thereof and its use in the treatment of plaque psoriasis of the scalp. In some embodiments, provided herein is a method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; and wherein hum13B8-b comprises: (i) a light chain polypeptide comprising the amino acid sequence of SEQ ID NO: 1; and a heavy chain polypeptide comprising the amino acid sequence of SEQ ID NO: 2.

In one embodiment, the present disclosure provides use of an anti-IL-23p19 antibody hum13B8-b for the manufacture of a medicament for treating plaque psoriasis of the scalp in a patient; wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein a first dose of the medicament is subcutaneously administered to the patient on week 0, a second dose of the medicament is subcutaneously administered to the patient at 4 weeks, and a subsequent dose of the medicament is subcutaneously administered to the patient every 4 to 12 weeks thereafter.

In a further embodiment the present disclosure provides the use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in an Investigator Global Assessment (IGA) mod 2011 rating scale (Scalp) score of “clear” or “almost clear” with at least a 2-point reduction from the patient's baseline score.

In a further embodiment the present disclosure provides a method of treating psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

In a further embodiment the present disclosure provides a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) score for the scalp of “clear” or “almost clear” with at least a 2-point reduction from Baseline at Week 16.

In a further embodiment the present disclosure provides a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) score for the scalp of “clear” or “almost clear” with at least a 2-point reduction from Baseline at Week 12.

In a further embodiment the present disclosure provides a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) Scalp Response Rate of 0 (clear) or 1 (almost clear) at Week 12.

In a further embodiment the present disclosure provides a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving Psoriasis Scalp Severity Index (PSSI) 90 at Week 16.

In a further embodiment the present disclosure provides a method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving Psoriasis Scalp Severity Index (PSSI) 90 at Week 12.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the CH3 domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antigen binding fragment” as used herein refers to a portion of an intact antibody and/or refers to the antigenic determining variable domains of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In particular embodiments, the anti-IL-23p19 antibody hum13B8-b is tildrakizumab. The term “tildrakizumab” as used herein refers to a humanized anti-IL-23p19 monoclonal antibody, also known as SCH 900222 or MK-3222. Tildrakizumab is a high-affinity (297 picomolar [pM]) humanized immunoglobulin G1/kappa (IgG1/x) antibody that specifically binds to the p19 protein of the IL-23 heterodimer, but does not bind human IL-12 (IL-12/23p40 and IL12p35 heterodimer) or human IL-12/23p40. Pharmacokinetics: Tildrakizumab pharmacokinetics increases proportionally over a dose range from 50 mg to 200 mg (0.5 to 2 times the approved recommended dosage) following subcutaneous administration in subjects with plaque psoriasis. Steady-state concentrations were achieved by Week 16 following subcutaneous administration of tildrakizumab at Weeks 0, 4, and every 12 weeks thereafter. At the 100 mg dose at Week 16, the mean (±SD) steady-state trough concentrations ranged from 1.22±0.94 mcg/mL to 1.47±1.12 mcg/mL. The geometric mean (CV %) steady-state Cmax was 8.1 mcg/mL (34%). The absolute bioavailability of tildrakizumab was estimated to be 73-80% following subcutaneous injection. The peak concentration (Cmax) was reached by approximately 6 days.

In some embodiments, the anti-IL-23p19 antibody tildrakizumab can refer to ILUMYA®. ILUMYA® is administered by subcutaneous injection at a recommended dosage of 100 mg at weeks 0, 4, and every 12 weeks thereafter. In some embodiments, tildrakizumab is formulated in a 1 mL single-dose prefilled syringe containing 100 mg of tildrakizumab (i.e., 100 mg/mL). In some embodiments, ILUMYA® (tildrakizumab-asmn) injection, for subcutaneous use, is a sterile, clear to slightly opalescent, colorless to slightly yellow solution. ILUMYA® is supplied in a single-dose prefilled syringe with a glass barrel and 29-gauge fixed, ½-inch needle. In some embodiments, tildrakizumab can be formulated in: L-histidine, L-histidine hydrochloride monohydrate, polysorbate 80, and/or sucrose, in Water for Injection, with a pH of 5.7-6.3. In some embodiments, tildrakizumab is formulated in a 1 mL single-dose prefilled syringe containing 100 mg of tildrakizumab-asmn formulated in: L-histidine (0.495 mg), L-histidine hydrochloride monohydrate (1.42 mg), polysorbate 80 (0.5 mg), sucrose (70.0 mg), and Water for Injection, USP with a pH of 5.7-6.3.

In particular embodiments, tildrakizumab comprises a light chain polypeptide comprising the amino acid sequence of SEQ ID NO: 1 and a heavy chain polypeptide comprising the amino acid sequence of SEQ ID NO: 2, and which is disclosed in U.S. Pat. Nos. 8,404,813 and 8,293,883, the disclosures of each of which are hereby incorporated by reference in their entireties. In other embodiments, tildrakizumab or an antigen-binding fragment thereof comprises a heavy chain variable domain and a light chain variable domain, wherein the heavy chain variable domain comprises CDR1, CDR2, and CDR3 sequences of the amino acid sequences of SEQ ID NOs: 3-5, and wherein the light chain variable domain comprises CDR1, CDR2, and CDR3 sequences of the amino acid sequences of SEQ ID NOs: 6-8.

As used herein, the term “subject” and “patient” are interchangeable. In some embodiments, subjects and/or patients are mammals.

A “disorder” is any condition that would benefit from treatment using the antibodies of the disclosure. “Disorder” and “condition” are used interchangeably herein and include chronic and acute disorders or diseases, including those pathological conditions that predispose a patient to the disorder in question.

The terms “treatment” or “treat” as used herein refer to both therapeutic treatment and prophylactic or preventative measures. Those in need of treatment include patients having psoriasis of the scalp as well as those prone to have psoriasis of the scalp or those in which psoriasis of the scalp is to be prevented.

The terms “administration” or “administering” as used herein refer to providing, contacting, and/or delivering an antibody or fragment thereof by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants. In an embodiment, administration is subcutaneous via a pre-filled syringe (PFS).

In some embodiments, the anti-IL-23p19 antibody hum13B8-b or an antigen-binding fragment thereof is administered every two weeks, every four weeks, every six weeks, every eight weeks, every ten weeks, or every twelve weeks.

As used herein, the term “Week 0” refers to the first day the anti-IL-23p19 antibody hum13B8-b or an antigen-binding fragment thereof is administered.

In some embodiments, the anti-IL-23p19 antibody hum13B8-b or an antigen-binding fragment thereof is administered over a two week treatment period, over a four week treatment period, over a six week treatment period, over an eight week treatment period, over a twelve week treatment period, over a sixteen week treatment period, over a twenty week treatment period, over a twenty-four week treatment period, over a twenty-eight week treatment period, over a thirty-two week treatment period, over a thirty-six week treatment period, over a forty-eight week treatment period, over a fifty-two week treatment period, over a sixty week treatment period, over a seventy-two week treatment period, or over a one year or more treatment period.

The therapy dose or therapeutically effective amount of the anti-IL-23p19 antibody hum13B8-b or an antigen-binding fragment thereof will vary depending, in part, upon the size (body weight, body surface, or organ size) and condition (the age and general health) of the patient. In some embodiments, the patient is administered one or more doses of the anti-IL-23p19 antibody hum13B8-b or an antigen-binding fragment thereof, wherein the dose is about 20 mg, 40 mg, 60 mg, 80 mg, 100 mg, 120 mg, 140 mg, 160 mg, 180 mg, or 200 mg. In some embodiments, the first dose, the second dose and the subsequent dose of the anti-IL-23p19 antibody hum13B8-b or an antigen-binding fragment thereof are the same. In some embodiments, the first dose, the second dose, and the subsequent dose of the anti-IL-23p19 antibody hum13B8-b or an antigen-binding fragment thereof are different. In some embodiments, the first dose is 100 mg. In some embodiments, the first dose is 200 mg. In some embodiments, the second dose is 100 mg. In some embodiments, the second dose is 200 mg. In some embodiments, the subsequent dose is 100 mg. In some embodiments, the subsequent dose is 200 mg. In some embodiments, the first dose, the second dose, and the subsequent dose are 100 mg. In some embodiments, the first dose, the second dose, and the subsequent dose are 200 mg. In some embodiments, the first dose, the second dose, and the subsequent dose contain 100 mg hum13B8-b. In some embodiments, the first dose, the second dose, and the subsequent dose contain 200 mg hum13B8-b.

The term “therapeutically effective amount” as applied to dose or amount refers to the quantity of a compound or pharmaceutical composition that is sufficient to result in a desired effect upon administration to a patient in need thereof. As used herein with respect to the pharmaceutical compositions comprising the anti-IL-23p19 antibody hum13B8-b (i.e., tildrakizumab), the term “therapeutically effective amount” also refers to the dose of a compound or pharmaceutical composition that is sufficient to produce an effective response upon administration to a patient. In some embodiments, a therapeutically effective amount of the anti-IL-23p19 antibody hum13B8-b (i.e., tildrakizumab) refers to an amount required to result in a significant improvement of psoriasis of the scalp of as assessed by one or more of Investigator Global Assessment (IGA) mod 2011 rating scale (Scalp), Investigator Global Assessment (Scalp only), Scalp Itch Numeric Rating Scale, Scalp Surface Area, Psoriasis Scalp Severity Index, Investigator Global Assessment (IGA) mod 2011 rating scale (Whole body), Physician Global Assessment of Skin (Whole body), Psoriasis Area and Severity Index, and/or Body Surface Area and Dermatology Life Quality Index. In some embodiments, a therapeutically effective amount of the anti-IL-23p19 antibody hum13B8-b (i.e., tildrakizumab) refers to a dose of 20 mg, 40 mg, 60 mg, 80 mg, 100 mg, 120 mg, 140 mg, 160 mg, 180 mg, or 200 mg. In some embodiments, a therapeutically effective amount of the anti-IL-23p19 antibody hum13B8-b (i.e., tildrakizumab) is a dose of 100 mg. In some embodiments, a therapeutically effective amount of the anti-IL-23p19 antibody hum13B8-b (i.e., tildrakizumab) is a dose of 100 mg at weeks 0, 4, and every 12 weeks thereafter.

In various embodiments, the assessments of psoriasis of the scalp include, but are not limited to Investigator Global Assessment (IGA) mod 2011 rating scale (Scalp), Investigator Global Assessment (Scalp only), Scalp Itch Numeric Rating Scale, Scalp Surface Area, Psoriasis Scalp Severity Index, Investigator Global Assessment (IGA) mod 2011 rating scale (Whole body), Physician Global Assessment of Skin (Whole body), Psoriasis Area and Severity Index, and/or Body Surface Area and Dermatology Life Quality Index.

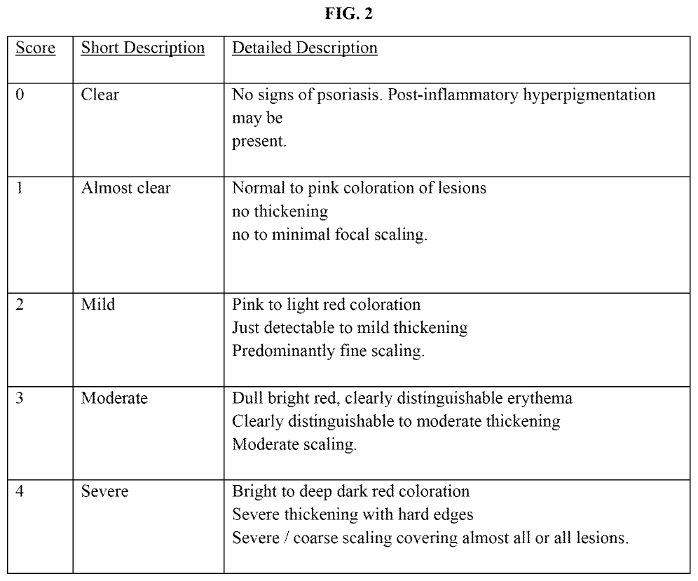

Investigator Global Assessment (IGA) mod 2011 rating scale (Scalp) or “IGA mod 2011” refers to a 5-point scale that is static (refers exclusively to the subject's disease state at the time of the assessments and does not attempt a comparison with any of the subject's previous disease states, whether at baseline or at a previous visit) (Langley et al., J. Dermatol. Treat. 26(1): 23-31 (2015)) (see FIG. 2). In some embodiments, a significant improvement of psoriasis of the scalp as assessed by Investigator Global Assessment (IGA) mod 2011 rating scale (Scalp) can refer to an IGA mod 2011 (scalp) score of “clear” or “almost clear” with at least a 2-point reduction from Baseline to Week 16, 28, 40, or 52. In some embodiments, a significant improvement can refer to patients with an IGA mod 2011 (scalp) score of “clear” with at least 2-point reduction from Baseline to Week 16, 28, 40, or 52. In some embodiments, a significant improvement of psoriasis of the scalp as assessed by Investigator Global Assessment (IGA) mod 2011 rating scale (Scalp) can refer to an IGA mod 2011 (scalp) score of “clear” or “almost clear” with at least a 2-point reduction from Baseline to Week 16, 18, 20, 22, 24, 26, 28, 30, 32, 34, 36, 38, 40, 42, 44, 46, 48, 50, or 52. In some embodiments, a significant improvement can refer to patients with an IGA mod 2011 (scalp) score of “clear” with at least 2-point reduction from Baseline to Week 16, 18, 20, 22, 24, 26, 28, 30, 32, 34, 36, 38, 40, 42, 44, 46, 48, 50, or 52.

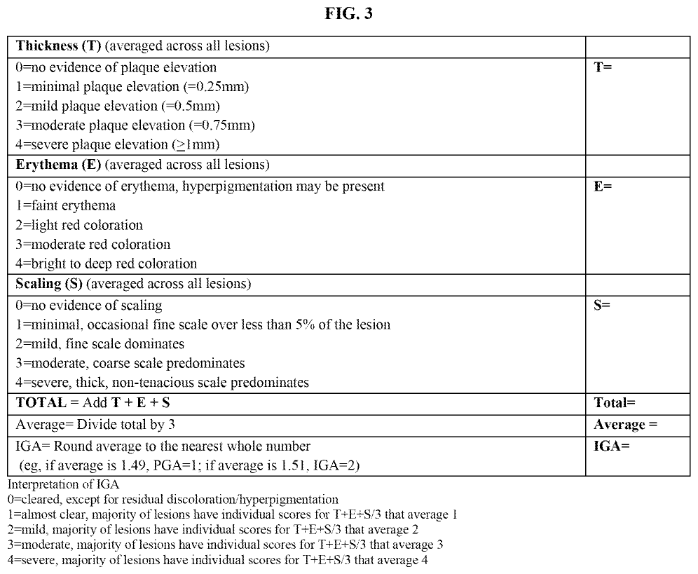

Investigator Global Assessment (Scalp only) refers to a 5-point IGA score and is a useful clinician assessment of scalp lesions based on thickness, erythema, and scaling. The scale defines the score as 0=clear; 1=almost clear; 2=mild; 3=moderate; 4=severe (see FIG. 3). In some embodiments, a significant improvement of psoriasis of the scalp as assessed by Investigator Global Assessment (IGA) scale (Scalp only) can refer to subjects with IGA mod 2011 score (whole body) score of “clear” or “almost clear” with at least a 2-point reduction from Baseline to Week 16, 28, 40, or 52. In some embodiments, a significant improvement of psoriasis of the scalp as assessed by Investigator Global Assessment (IGA) scale (Scalp only) can refer to subjects with IGA mod 2011 score (whole body) score of “clear” or “almost clear” with at least a 2-point reduction from Baseline to Week 16, 18, 20, 22, 24, 26, 28, 30, 32, 34, 36, 38, 40, 42, 44, 46, 48, 50, or 52.

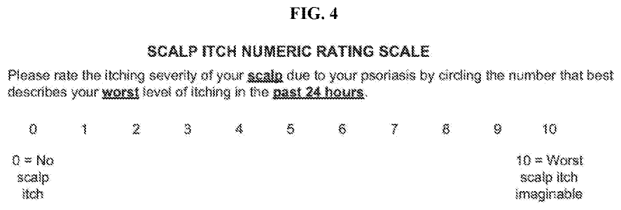

Scalp Itch Numeric Rating Scale (Scalp Itch NRS) refers to a self-administered, single item questionnaire with response options from 0=“No Itch” to 10=“Worst itch imaginable”. A higher score on the NRS corresponds to greater scalp itch severity (see FIG. 4). In some embodiments, a significant improvement of psoriasis of the scalp as assessed by scalp itch NRS can refer to subjects achieving at least a 3-point reduction In some embodiments, a significant improvement of psoriasis of the scalp as assessed by scalp itch NRS can refer to subjects achieving a 5-point reduction in Scalp Itch NRS score from Baseline to Week 16, 28, 40, or 52. In some embodiments, a significant improvement of psoriasis of the scalp as assessed by scalp itch NRS can refer to subjects achieving a 6-point reduction in Scalp Itch NRS score from Baseline to Week 16, 28, 40, or 52. In some embodiments, a significant improvement of psoriasis of the scalp as assessed by scalp itch NRS can refer to subjects achieving a 3-point, 4-point, 5-point, or 6-point reduction in Scalp Itch NRS score from Baseline to Week 16, 18, 20, 22, 24, 26, 28, 30, 32, 34, 36, 38, 40, 42, 44, 46, 48, 50, or 52.

Scalp Surface Area (SSA) refers to the scalp surface area measured using the palm method where the palm of the subject's hand (including the palmar aspect of the fingers) represents 20% of the SSA. The affected areas are then calculated by their size compared to the subject's palm. In some embodiments, a significant improvement of psoriasis of the scalp as assessed by SSA can refer to at least a 5%, at least a 10%, at least a 20%, at least a 25%, at least a 30%, at least a 40%, at least a 50%, or at least a 75%, or more decrease in the scalp surface area affected from Baseline to Week 16, 28, 40, or 52. In some embodiments, a significant improvement of psoriasis of the scalp as assessed by SSA can refer to at least a 5%, at least a 10%, at least a 20%, at least a 25%, at least a 30%, at least a 40%, at least a 50%, or at least a 75%, or more decrease in the scalp surface area affected from Baseline to Week 16, 18, 20, 22, 24, 26, 28, 30, 32, 34, 36, 38, 40, 42, 44, 46, 48, 50, or 52.

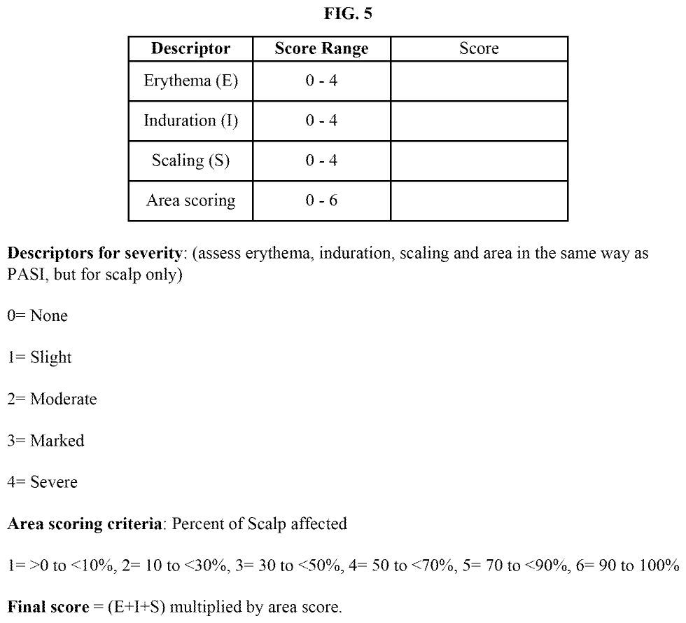

Psoriasis Scalp Severity Index (PSSI) refers to a modification of the PASI, to specifically assess severity of scalp disease. The PSSI uses a 5-point scale to grade the 3 clinical parameters (erythema, thickness, and scaling) in the same way as the PASI, but for scalp only. The parameter scores are summed and multiplied by an integer (0-6) that represents the area of affected scalp (see FIG. 5). In some embodiments, a significant improvement of psoriasis of the scalp as assessed by PSSI can refer to a subject achieving at least a 75% improvement in the Psoriasis Scalp Severity Index (PSSI) score from Baseline to Week 16, 28, 40, or 52. In some embodiments, a significant improvement of psoriasis of the scalp as assessed by PSSI can refer to a subject achieving at least a 90% improvement in the Psoriasis Scalp Severity Index (PSSI) score from Baseline to Week 16, 28, 40, or 52. In some embodiments, a significant improvement of psoriasis of the scalp as assessed by PSSI can refer to a subject achieving at least a 100% improvement in the Psoriasis Scalp Severity Index (PSSI) score from Baseline to Week 16, 28, 40, or 52. In some embodiments, a significant improvement of psoriasis of the scalp as assessed by PSSI can refer to a subject achieving at least a 75%, 90%, or 100% improvement in the Psoriasis Scalp Severity Index (PSSI) score from Baseline to Week 16, 18, 20, 22, 24, 26, 28, 30, 32, 34, 36, 38, 40, 42, 44, 46, 48, 50, or 52.

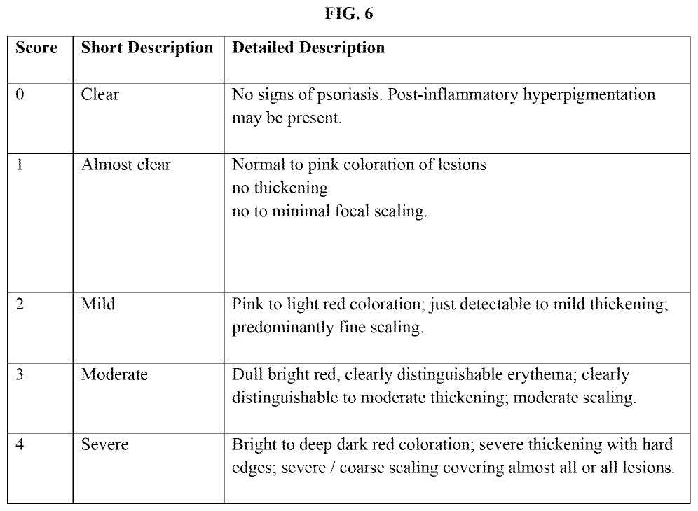

The Investigator Global Assessment (IGA) mod 2011 rating scale (whole body) refers to a 5-point scale that is static (refers exclusively to the subject's disease state at the time of the assessments, and does not attempt a comparison with any of the subject's previous disease states, whether at baseline or at a previous visit) (Langley, 2015) (see FIG. 6). In some embodiments, a significant improvement of psoriasis of the scalp as assessed by Investigator Global Assessment (IGA) mod 2011 rating scale (whole body) can refer to refer to an IGA mod 2011 (whole body) score of “clear” or “almost clear” with at least a 2-point reduction from Baseline to Week 16, 28, 40, or 52. In some embodiments, a significant improvement can refer to patients with an IGA mod 2011 (whole body) score of “clear” with at least 2-point reduction from Baseline to Week 16, 28, 40, or 52. In some embodiments, a significant improvement of psoriasis of the scalp as assessed by Investigator Global Assessment (IGA) mod 2011 rating scale (whole body) can refer to refer to an IGA mod 2011 (whole body) score of “clear” or “almost clear” with at least a 2-point reduction from Baseline to Week 16, 18, 20, 22, 24, 26, 28, 30, 32, 34, 36, 38, 40, 42, 44, 46, 48, 50, or 52. In some embodiments, a significant improvement can refer to patients with an IGA mod 2011 (whole body) score of “clear” with at least 2-point reduction from Baseline to Week 16, 18, 20, 22, 24, 26, 28, 30, 32, 34, 36, 38, 40, 42, 44, 46, 48, 50, or 52.

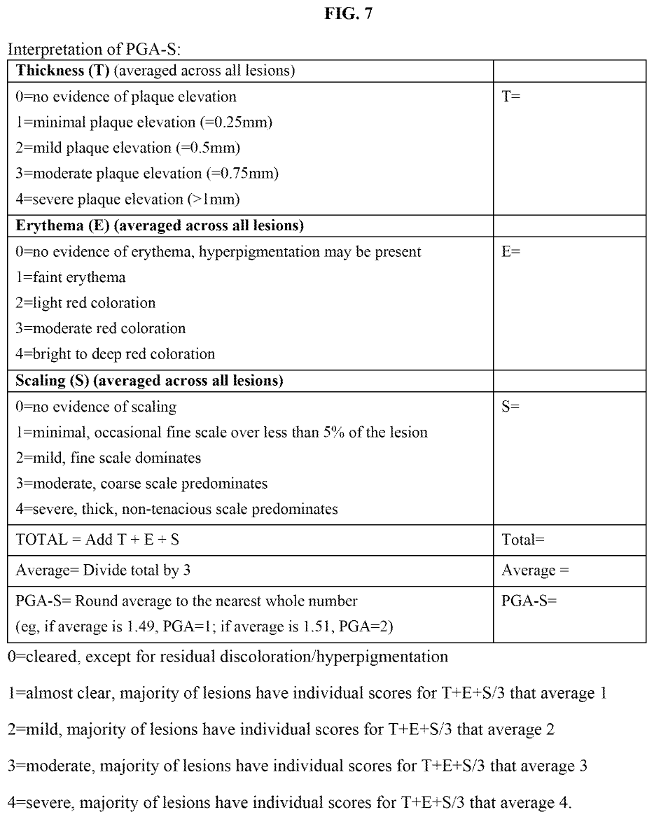

Physician Global Assessment (PGA) of Skin (Whole body) refers to a 5-point, 0-4 measure that is a useful clinician assessment of psoriasis lesions on the skin based on degree of erythema, thickness, and scale averaged over the entire body. Each of the clinical signs are assessed on a 0-5 scale (0=clear, 1=minimal, 2=mild, 3=moderate, and 4=severe) (see FIG. 7). In some embodiments, a significant improvement of psoriasis of the scalp as assessed by Physician Global Assessment of Skin (Whole body) can refer to subjects with a PGA of skin (whole body) score of “clear” or “almost clear” with at least a 2-point reduction from Baseline to Week 16, 28, 40, or 52. In some embodiments, a significant improvement of psoriasis of the scalp as assessed by Physician Global Assessment of Skin (Whole body) can refer to subjects with a PGA of skin (whole body) score of “clear” with at least a 2-point reduction from Baseline to Week 16, 28, 40, or 52. In some embodiments, a significant improvement of psoriasis of the scalp as assessed by Physician Global Assessment of Skin (Whole body) can refer to subjects with a PGA of skin (whole body) score of “clear” or “almost clear” with at least a 2-point reduction from Baseline to Week 16, 18, 20, 22, 24, 26, 28, 30, 32, 34, 36, 38, 40, 42, 44, 46, 48, 50, or 52. In some embodiments, a significant improvement of psoriasis of the scalp as assessed by Physician Global Assessment of Skin (Whole body) can refer to subjects with a PGA of skin (whole body) score of “clear” with at least a 2-point reduction from Baseline to Week 16, 18, 20, 22, 24, 26, 28, 30, 32, 34, 36, 38, 40, 42, 44, 46, 48, 50, or 52.

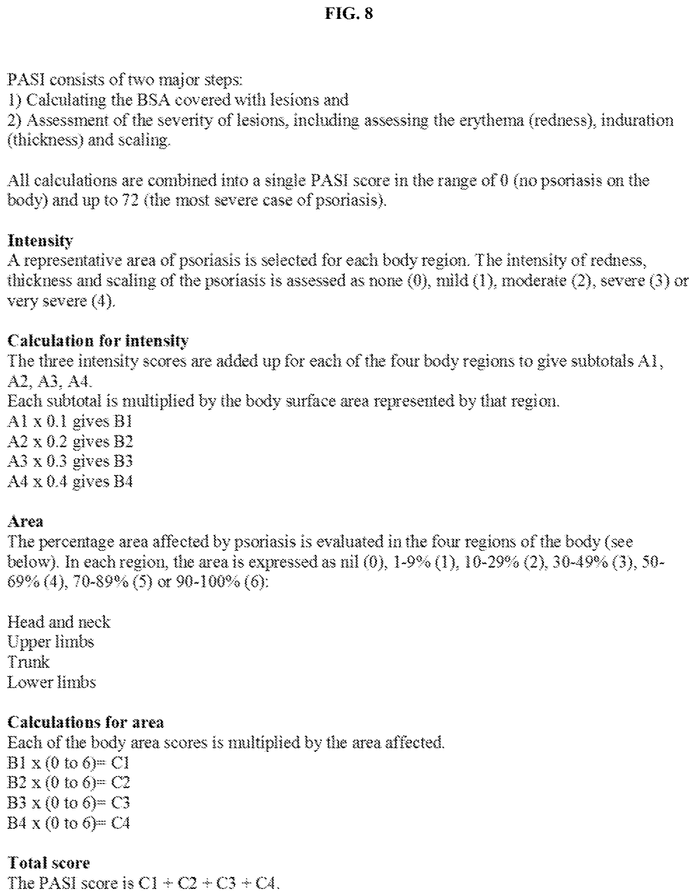

Psoriasis Area and Severity Index (PASI) is used to determine the treatment response (PASI 75, PASI 90, and PASI 100) in subjects with scalp psoriasis. The PASI includes scores on erythema, thickness, scaling, and percentage of body surface area (BSA) affected (see FIG. 8). In some embodiments, a significant improvement of psoriasis of the scalp as assessed by PASI can refer to a subject achieving at least a 75% improvement in the PASI score from Baseline to Week 16, 28, 40, or 52. In some embodiments, a significant improvement of psoriasis of the scalp as assessed by PASI can refer to a subject achieving at least a 90% improvement in the PASI score from Baseline to Week 16, 28, 40, or 52. In some embodiments, a significant improvement of psoriasis of the scalp as assessed by PASI can refer to a subject achieving at least a 100% improvement in the PASI score from Baseline to Week 16, 28, 40, or 52. In some embodiments, a significant improvement of psoriasis of the scalp as assessed by PASI can refer to a subject achieving at least a 75%, 90%, or 100% improvement in the PASI score from Baseline to Week 16, 18, 20, 22, 24, 26, 28, 30, 32, 34, 36, 38, 40, 42, 44, 46, 48, 50, or 52.

Body Surface Area is a commonly used measure of severity of skin disease. It is defined as the percentage of the total BSA affected by psoriasis, it represents the area of affected scalp. The BSA will be measured using the palm method where the palm of the subject's hand (including the palmar aspect of the fingers) represents 1% of the BSA. The affected areas are then calculated by their size compared to the subject's palm. In some embodiments, a significant improvement of psoriasis of the scalp as assessed by body surface area can refer to at least a 5%, at least a 10%, at least a 20%, at least a 25%, at least a 30%, at least a 40%, at least a 50%, or at least a 75%, or more decrease in the body surface area affected from Baseline to Week 16, 28, 40, or 52. In some embodiments, a significant improvement of psoriasis of the scalp as assessed by body surface area can refer to at least a 5%, at least a 10%, at least a 20%, at least a 25%, at least a 30%, at least a 40%, at least a 50%, or at least a 75%, or more decrease in the body surface area affected from Baseline to Week 16, 18, 20, 22, 24, 26, 28, 30, 32, 34, 36, 38, 40, 42, 44, 46, 48, 50, or 52.

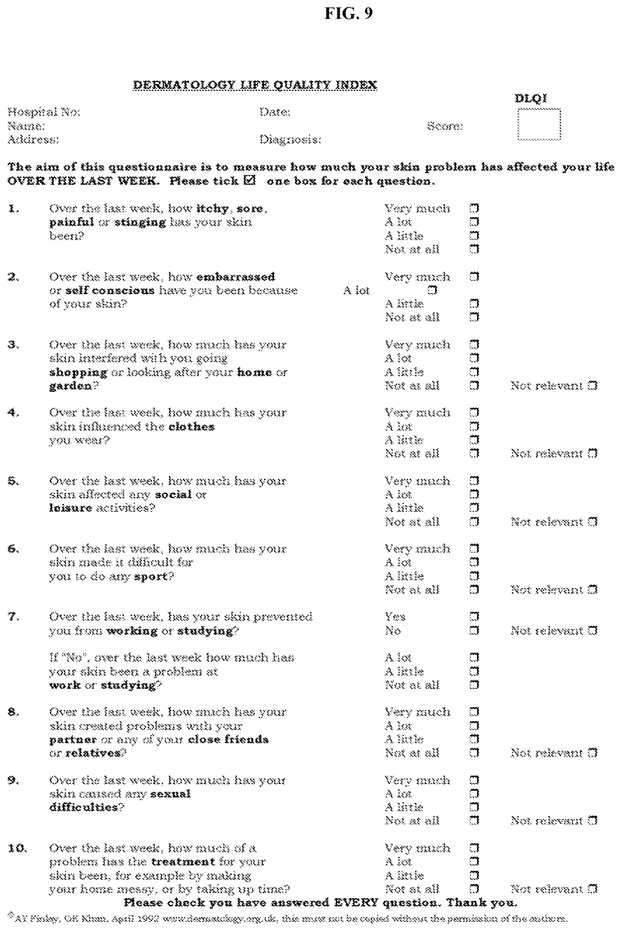

The Dermatology Life Quality Index (DLQI) questionnaire is used to assess treatment response on the subject's quality of life. DLQI is a 10-item questionnaire that measures the impact of skin disease on participant's quality of life. Each question was evaluated on a 4-point scale ranging from 0 (not at all) to 3 (very much), where higher scores indicate more impact on quality of life. Total scores range from 0 (not at all) to 30 (very much. A score of 0-1=no effect at all on the participant's life; a score of 2-6=small effect on the participant's life; a score of 7-12=moderate effect on the participant's life; a score of 13-18=very large effect on the participant's life; a score of 19-30=extremely large effect on the participant's life. Higher scores indicate more impact on quality of life of participants. The aim of this questionnaire is to measure how much the skin condition has affected the subject's life during the previous week. Subjects are asked to recall their experiences during the previous week by responding to 10 questions (see FIG. 9). In some embodiments, a significant improvement of psoriasis of the scalp as assessed by DLQI can refer to a positive change of 2, 3, 4, 5, 6, 7, 8, 9, or 10 or more in the DLQI score from Baseline to Week 16, 28, 40, or 52.

As used herein, the term “significant improvement” refers to significant positive effect in a response in patients taking the anti-IL-23p19 antibody hum13B8-b containing the anti-IL-23p19 antibody hum13B8-b (i.e., tildrakizumab) relative to patients taking a placebo. In some embodiments, a significant improvement of psoriasis of the scalp can be as assessed by one or more of the following: Investigator Global Assessment (IGA) mod 2011 rating scale (Scalp), Investigator Global Assessment (Scalp only), Scalp Itch Numeric Rating Scale, Scalp Surface Area, Psoriasis Scalp Severity Index, Investigator Global Assessment (IGA) mod 2011 rating scale (Whole body), Physician Global Assessment of Skin (Whole body), Psoriasis Area and Severity Index, and/or Body Surface Area and Dermatology Life Quality Index. In certain embodiments, the significant improvement refers to a statistically significant improvement. In certain embodiments, the term “statistically significant” means having a probability of less than 10% under the relevant null hypothesis (i.e., p<0.1). In some embodiments, a p-value less than 0.05 is considered to be statistically significant. In some embodiments, a p-value less than 0.01 is considered to be statistically significant. In some embodiments, a p-value less than 0.005 is considered to be statistically significant. In some embodiments, a p-value less than 0.0025 is considered to be statistically significant. In some embodiments, a p-value less than 0.001 is considered to be statistically significant. In certain embodiments, statistical tests will be 2-sided at the 5% significance level, and point estimates are accompanied with 2-sided 95% confidence intervals (CIs), where applicable.

In some embodiments, a significant improvement can refer to an improvement of psoriasis of the scalp of at least 10%, 15%, 20%, 25%, 30%, 35%, 40%, 45%, 50%, 55%, 60%, 65%, 70%, 75%, 80%, 85%, 90%, 95%, 100%, 150%, or 200% or more as assessed by one or more of Investigator Global Assessment (IGA) mod 2011 rating scale (Scalp), Investigator Global Assessment (Scalp only), Scalp Itch Numeric Rating Scale, Scalp Surface Area, Psoriasis Scalp Severity Index, Investigator Global Assessment (IGA) mod 2011 rating scale (Whole body), Physician Global Assessment of Skin (Whole body), Psoriasis Area and Severity Index, and/or Body Surface Area and Dermatology Life Quality Index.

In some embodiments, a significant improvement can refer to an at least 2-fold, 3-fold, 4-fold, 5-fold, or 10-fold, or more than 10-fold improvement of psoriasis of the scalp of as assessed by one or more of Investigator Global Assessment (IGA) mod 2011 rating scale (Scalp), Investigator Global Assessment (Scalp only), Scalp Itch Numeric Rating Scale, Scalp Surface Area, Psoriasis Scalp Severity Index, Investigator Global Assessment (IGA) mod 2011 rating scale (Whole body), Physician Global Assessment of Skin (Whole body), Psoriasis Area and Severity Index, and/or Body Surface Area and Dermatology Life Quality Index.

In some embodiments, provided herein is a method of treating plaque psoriasis of the scalp comprising administering a therapeutically effective amount of an anti-IL-23p19 antibody hum13B8-b to a patient in need thereof, wherein administration of hum13B8-b results in an Investigator Global Assessment (IGA) mod 2011 rating scale (Scalp) score of “clear” or “almost clear” with at least a 2-point reduction from the patient's baseline score; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

In some embodiments, provide herein is a method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter, and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

In further embodiments, provided herein is use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in an Investigator Global Assessment (IGA) mod 2011 rating scale (Scalp) score of “clear” or “almost clear” with at least a 2-point reduction from the patient's baseline score.

In other embodiments, the use of the medicament further results in an improvement from the patient's baseline score for at least two of four parameters selected from the group consisting of (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

In other embodiments the use of the medicament further results in at least 90% improvement from the patient's baseline score for the Psoriasis Scalp Severity Index (PSSI).

In a further embodiment, the present disclosure provides the use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in an Investigator Global Assessment (IGA) mod 2011 rating scale (Scalp) score of “clear” or “almost clear” with at least a 2-point reduction from the patient's baseline score.

In some embodiments provided herein is a method of treating plaque psoriasis of the scalp comprising administering a therapeutically effective amount of an anti-IL-23p19 antibody hum13B8-b to a patient in need thereof, wherein administration of hum13B8-b results in a significant improvement from the patient's baseline score in the Investigator Global Assessment (IGA) mod 2011 rating scale (Scalp) score; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

In further embodiments, provided herein is use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2, and wherein administration of the medicament results in a significant improvement from the patient's baseline score in the Investigator Global Assessment (IGA) mod 2011 rating scale (Scalp) score.

In other embodiments the use of the medicament further results in a significant improvement from the patient's baseline score for at least two of the four parameters selected from the group consisting of (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

In other embodiments the use of the medicament further results in at least about 70%, 75%, 80%, 85%, 90%, 95%, 100%, 150%, or 200% or more improvement from the patient's baseline score for at least two of the four parameters selected from the group consisting of (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

In other embodiments the use of the medicament further results in at least 90% improvement from the patient's baseline score for the Psoriasis Scalp Severity Index (PSSI).

In a further embodiment the present invention provides the use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in a significant improvement from the patient's baseline score in the Investigator Global Assessment (IGA) mod 2011 rating scale (Scalp) score.

In some embodiments provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a IGA mod 2011 (scalp) score of “clear” or “almost clear” with at least a 2-point reduction from patient's baseline score at week 12, or week 16, or week 20, or week 24, or week 28, or week 32, or week 36, or week 40, or week 44, or week 48, or week 52 indicates the efficacy of hum13B8-b.

In some embodiments provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an IGA (Scalp) score of “clear” and “almost clear” with at least a 2-point reduction from patient's baseline score at week 52 indicates the efficacy of the antibody.

In some embodiments provided herein is a method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a 4-point reduction in Scalp Itch NRS score from patient's baseline score at week 16 indicates the efficacy of the antibody.

In some embodiments provided herein is a method of determining the efficacy of an anti-IL-23p19 antibody for the treatment of plaque psoriasis of the scalp comprising administering a therapeutically effective amount an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a PGA-S score (whole body) of “clear” or “almost clear” with at least a 2-point reduction from the patient's baseline score at week 16 indicates the efficacy of the antibody.

The terms “pharmaceutical composition” or “therapeutic composition” as used herein refer to a compound or composition capable of inducing a desired therapeutic effect when properly administered to a patient. One embodiment of the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier” as used herein refer to one or more formulation materials suitable for accomplishing or enhancing the delivery of one or more antibodies of the disclosure.

Pharmaceutical compositions comprising tildrakizumab, either alone or in combination with prophylactic agents, therapeutic agents, and/or pharmaceutically acceptable carriers are provided. The pharmaceutical compositions comprising tildrakizumab provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art.

In some embodiments provided herein is a pharmaceutical composition of an anti-IL-23p19 antibody hum13B8-b for the treatment of psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

In some embodiments provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter.

In some embodiments provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in an Investigator Global Assessment (IGA) mod 2011 rating scale (Scalp) score of “clear” or “almost clear” with at least a 2-point reduction from the patient's baseline score.

In some embodiments provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in a significant improvement from the patient's baseline score in an Investigator Global Assessment (IGA) mod 2011 rating scale (Scalp) score.

In some embodiments provided herein is a pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter, and wherein administration of the pharmaceutical composition results in an Investigator Global Assessment (IGA) mod 2011 rating scale (Scalp) score of “clear” or “almost clear” with at least a 2-point reduction from the patient's baseline score.

In some embodiments provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) score for the scalp of “clear” or “almost clear” with at least a 2-point reduction from Baseline at Week 16.

In some embodiments provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) score for the scalp of “clear” or “almost clear” with at least a 2-point reduction from Baseline at Week 12.

In some embodiments provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) Scalp Response Rate of 0 (clear) or 1 (almost clear) at Week 12.

In some embodiments provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the antibody results in the patient achieving Psoriasis Scalp Severity Index (PSSI) 90 at Week 16.

In some embodiments provided herein is a pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving Psoriasis Scalp Severity Index (PSSI) 90 at Week 12.

### Embodiments

Embodiment 1: A method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; and wherein hum13B8-b comprises: (i) a light chain polypeptide comprising the amino acid sequence of SEQ ID NO: 1; and a heavy chain polypeptide comprising the amino acid sequence of SEQ ID NO: 2.

Embodiment 2: The method according to embodiment 1, wherein the first dose, the second dose, and the subsequent dose are the same.

Embodiment 3: The method according to embodiment 2, wherein the first dose, the second dose, and the subsequent dose are 100 mg.

Embodiment 4: The method according to embodiment 1, wherein the subsequent dose is administered to the patient every 12 weeks at least up to 16 weeks.

Embodiment 5: The method according to embodiment 1, wherein the subsequent dose is administered to the patient every 4 weeks at least up to 28 weeks.

Embodiment 6: The method according to embodiment 1, wherein the subsequent dose is administered to the patient every 12 weeks at least up to 40 weeks, at least up to 52 weeks, at least up to 72 weeks, or longer.

Embodiment 7: A method of treating plaque psoriasis of the scalp comprising administering a therapeutically effective amount of an anti-IL-23p19 antibody hum13B8-b to a patient in need thereof, wherein administration of hum13B8-b results in an Investigator Global Assessment (IGA) mod 2011 rating scale (Scalp) score of “clear” or “almost clear” with at least a 2-point reduction from the patient's baseline score; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

Embodiment 8: The method according to embodiment 7, wherein administration of hum13B8-b further results in a significant improvement from the patient's baseline score for at least two of four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

Embodiment 9: A method of treating plaque psoriasis of the scalp comprising administering a therapeutically effective amount of an anti-IL-23p19 antibody hum13B8-b to a patient in need thereof, wherein administration of hum13B8-b results in a significant improvement from the patient's baseline score in an Investigator Global Assessment (IGA) mod 2011 rating scale (Scalp) score; and wherein hum13B8-b comprises: (i) a light chain polypeptide comprising the amino acid sequence of SEQ ID NO: 1; and (ii) a heavy chain polypeptide comprising the amino acid sequence of SEQ ID NO: 2.

Embodiment 10: The method according to embodiment 9, wherein administration of humB13B-b further results in a significant improvement from the patient's baseline score for at least two of four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

Embodiment 11: A method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an Investigator Global Assessment (IGA) mod 2011 rating scale (Scalp) score of “clear” or “almost clear” with at least a 2-point reduction from the patient's baseline score at week 12, or week 16, or week 52 indicates the efficacy of the anti-IL-23p19 antibody.

Embodiment 12: The method according to embodiment 11, wherein an at least 90% improvement from the patient's baseline score for the Psoriasis Scalp Severity Index (PSSI) at week 12, or week 16, or week 52 further indicates the efficacy of the anti-IL-23p19 antibody.

Embodiment 13: A method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein administration of hum13B8-b results in an Investigator Global Assessment (IGA) mod 2011 rating scale (Scalp) score of “clear” or “almost clear” with at least a 2-point reduction from the patient's baseline score.

Embodiment 14: The method according to embodiment 13, wherein administration of hum13B8-b further results in a significant improvement from the patient's baseline score for the Psoriasis Scalp Severity Index (PSSI).

Embodiment 15: The method according to either embodiment 13 or 14, wherein the first dose, the second dose, and the subsequent dose are same.

Embodiment 16: The method according to embodiment 15, wherein the first dose, the second dose, and the subsequent dose are 100 mg.

Embodiment 17. A method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an Investigator Global Assessment (IGA) mod 2011 rating scale (Scalp) score of “clear” or “almost clear” with at least a 2-point reduction from the patient's baseline score at week 52 indicates the efficacy of the anti-IL-23p19 antibody.

Embodiment 18. The method according to embodiment 17, wherein a significant improvement from the patient's baseline score for the Psoriasis Scalp Severity Index (PSSI) at week 52 further indicates the efficacy of the anti-IL-23p19 antibody.

Embodiment 19. The method according to embodiment 17, wherein an at least 75% improvement from the patient's baseline score for the Psoriasis Scalp Severity Index (PSSI) at week 52 further indicates the efficacy of the anti-IL-23p19 antibody.

Embodiment 20. A method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a 4-point reduction in Scalp Itch NRS score from the patient's baseline score at week 16 indicates the efficacy of the anti-IL-23p19 antibody.

Embodiment 21. A method of determining the efficacy of an anti-IL-23p19 antibody for the treatment of plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; wherein hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a PGA-S score (whole body) of “clear” or “almost clear” with at least a 2-point reduction from the patient's baseline score at week 16 indicates the efficacy of the anti-IL-23p19 antibody.

Embodiment 22. Use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein a first dose of the medicament is subcutaneously administered to the patient on week 0, a second dose of the medicament is subcutaneously administered to the patient at 4 weeks, and a subsequent dose of the medicament is subcutaneously administered to the patient every 4 to 12 weeks thereafter.

Embodiment 23: The use according to embodiment 22, wherein the first dose of the medicament, the second dose of the medicament, and the subsequent dose of the medicament are the same.

Embodiment 24: The use according to embodiment 23, wherein the first dose of the medicament, the second dose of the medicament, and the subsequent dose of the medicament contain 100 mg hum13B8-b.

Embodiment 25: The use according to embodiment 22, wherein the subsequent dose of the medicament is administered every 12 weeks at least up to 16 weeks.

Embodiment 26: The use according to embodiment 22, wherein the subsequent dose of the medicament is administered every 4 weeks at least up to 28 weeks.

Embodiment 27: The use according to embodiment 22, wherein the subsequent dose of the medicament is administered every 12 weeks at least up to 40 weeks, at least up to 52 weeks, at least up to 72 weeks, or longer.

Embodiment 28: Use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in an Investigator Global Assessment (IGA) mod 2011 rating scale (Scalp) score of “clear” or “almost clear” with at least a 2-point reduction from the patient's baseline score.

Embodiment 29: The use according to embodiment 28, wherein administration of the medicament further results in a significant improvement from the patient's baseline score for at least two of four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

Embodiment 30: Use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in a significant improvement from the patient's baseline score in an Investigator Global Assessment (IGA) mod 2011 rating scale (Scalp) score.

Embodiment 31: The use according to embodiment 30, wherein administration of the medicament further results in a significant improvement from the patient's baseline score for at least two of the four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

Embodiment 32: Use of an anti-IL-23p19 antibody hum13B8-b for the manufacture of a medicament for treating plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is subcutaneously administered to the patient on week 0, a second dose of the medicament is subcutaneously administered to the patient at 4 weeks, and a subsequent dose of the medicament is subcutaneously administered to the patient every 4 to 12 weeks thereafter, and wherein administration of the medicament results in an Investigator Global Assessment (IGA) mod 2011 rating scale (Scalp) score of “clear” or “almost clear” with at least a 2-point reduction from the patient's baseline score.

Embodiment 33: The use according to embodiment 32, wherein administration of the medicament further results in a significant improvement from the patient's baseline score for the Psoriasis Scalp Severity Index (PSSI).

Embodiment 34: The use according to either embodiment 32 or 33, wherein the first dose of the medicament, the second dose of the medicament, and the subsequent dose of the medicament are same.

Embodiment 35: The use according to embodiment 34, wherein the first dose of the medicament, the second dose of the medicament, and the subsequent dose of the medicament contain 100 mg hum13B8-b.

Embodiment 36. A pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter.

Embodiment 37: The pharmaceutical composition according to embodiment 36, wherein the first dose of the pharmaceutical composition, the second dose of the pharmaceutical composition, and the subsequent dose of the pharmaceutical composition are the same.

Embodiment 38: The pharmaceutical composition according to embodiment 36, wherein the first dose of the pharmaceutical composition, the second dose of the pharmaceutical composition, and the subsequent dose of the pharmaceutical composition contain 100 mg hum13B8-b.

Embodiment 39: The pharmaceutical composition according to embodiment 36, wherein the subsequent dose of the pharmaceutical composition is administered every 12 weeks at least up to 16 weeks.

Embodiment 40: The pharmaceutical composition according to embodiment 36, wherein the subsequent dose of the pharmaceutical composition is administered every 4 weeks at least up to 28 weeks.

Embodiment 41: The pharmaceutical composition according to embodiment 36, wherein the subsequent dose of the pharmaceutical composition is administered every 12 weeks at least up to 40 weeks, at least up to 52 weeks, at least up to 72 weeks, or longer.

Embodiment 42: A pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in an Investigator Global Assessment (IGA) mod 2011 rating scale (Scalp) score of “clear” or “almost clear” with at least a 2-point reduction from the patient's baseline score.

Embodiment 43: The pharmaceutical composition according to embodiment 42, wherein administration of the pharmaceutical composition further results in a significant improvement from the patient's baseline score for at least two of four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

Embodiment 44: A pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in a significant improvement from the patient's baseline score in an Investigator Global Assessment (IGA) mod 2011 rating scale (Scalp) score.

Embodiment 45: The pharmaceutical composition according to embodiment 44, wherein administration of the pharmaceutical composition further results in a significant improvement from the patient's baseline score for at least two of the four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

Embodiment 46: A pharmaceutical composition of an anti-IL-23p19 antibody hum13B8-b for the treatment of plaque psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter, and wherein administration of the pharmaceutical composition results in an Investigator Global Assessment (IGA) mod 2011 rating scale (Scalp) score of “clear” or “almost clear” with at least a 2-point reduction from the patient's baseline score.

Embodiment 47: The pharmaceutical composition according to embodiment 46, wherein administration of the pharmaceutical composition further results in a significant improvement from the patient's baseline score for the Psoriasis Scalp Severity Index (PSSI).

Embodiment 48: The pharmaceutical composition according to either embodiment 46 or 47, wherein the first dose of the pharmaceutical composition, the second dose of the pharmaceutical composition, and the subsequent dose of the pharmaceutical composition are same.

Embodiment 49: The pharmaceutical composition according to embodiment 48, wherein the first dose of the pharmaceutical composition, the second dose of the pharmaceutical composition, and the subsequent dose of the pharmaceutical composition contain 100 mg hum13B8-b.

Embodiment 50: The use of an anti-IL-23p19 antibody hum13B8-b for the manufacture of a medicament for treating psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Embodiment 51: The use according to embodiment 50, wherein the psoriasis of the scalp is moderate to severe psoriasis of the scalp.

Embodiment 52: The use according to embodiment 50, wherein a first dose of the medicament is administered to the patient on week 0, a second dose of the medicament is administered to the patient at 4 weeks, and a subsequent dose of the medicament is administered to the patient every 4 to 12 weeks thereafter.

Embodiment 53: The use according to embodiment 52, wherein the first dose of the medicament, the second dose of the medicament, and the subsequent dose of the medicament are the same.

Embodiment 54: The use according to embodiment 53, wherein the first dose of the medicament, the second dose of the medicament, and the subsequent dose of the medicament contain 100 mg hum13B8-b.

Embodiment 55: The use according to embodiment 52, wherein the subsequent dose of the medicament is administered every 12 weeks at least up to 16 weeks.

Embodiment 56: The use according to embodiment 52, wherein the subsequent dose of the medicament is administered every 4 weeks at least up to 28 weeks.

Embodiment 57: The use according to embodiment 52, wherein the subsequent dose of the medicament is administered every 12 weeks at least up to 40 weeks, at least up to 52 weeks, at least up to 72 weeks, or longer.

Embodiment 58: The use according to embodiment 50, wherein the medicament is administered to the patient by subcutaneous injection.

Embodiment 59: The use according to embodiment 50, wherein the patient has an affected Scalp Surface Area (SSA) of at least 50%, a Psoriasis Area and Severity Index (PASI) score of at least 16.7, and/or a Physician Global Assessment (PGA) score of 3 (“moderate”) or 4 (“severe”).

Embodiment 60: The use according to embodiment 50, wherein the patient is male, White, and/or at least 44 years of age.

Embodiment 61: The use according to embodiment 50, wherein administration of the medicament results in the patient achieving an Investigator Global Assessment (IGA) score for the scalp of “clear” or “almost clear” with at least a 2-point reduction from Baseline at Week 16.

Embodiment 62: The use according to embodiment 50, wherein administration of the medicament results in the patient achieving an Investigator Global Assessment (IGA) score for the scalp of “clear” or “almost clear” with at least a 2-point reduction from Baseline at Week 12.

Embodiment 63: The use according to embodiment 50, wherein administration of the medicament results in the patient achieving an Investigator Global Assessment (IGA) Scalp Response Rate of 0 (clear) or 1 (almost clear) at Week 12.

Embodiment 64: The use according to embodiment 50, wherein administration of the medicament results in the patient achieving Psoriasis Scalp Severity Index (PSSI) 90 at Week 16.

Embodiment 65: The use according to embodiment 50, wherein administration of the medicament results in the patient achieving Psoriasis Scalp Severity Index (PSSI) 90 at Week 12.

Embodiment 66: The use according to an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) score for the scalp of “clear” or “almost clear” with at least a 2-point reduction from Baseline at Week 16.

Embodiment 67: The use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) score for the scalp of “clear” or “almost clear” with at least a 2-point reduction from Baseline at Week 12.

Embodiment 68: The use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) Scalp Response Rate of 0 (clear) or 1 (almost clear) at Week 12.

Embodiment 69: The use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving Psoriasis Scalp Severity Index (PSSI) 90 at Week 16.

Embodiment 70: The use of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving Psoriasis Scalp Severity Index (PSSI) 90 at Week 12.

Embodiment 71: A pharmaceutical composition of an anti-IL-23p19 antibody hum13B8-b for the treatment of psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Embodiment 72: The pharmaceutical composition according to embodiment 71, wherein the psoriasis of the scalp is moderate to severe psoriasis of the scalp.

Embodiment 73: The pharmaceutical composition according to embodiment 71, wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter.

Embodiment 74: The pharmaceutical composition according to embodiment 73, wherein the first dose of the pharmaceutical composition, the second dose of the pharmaceutical composition, and the subsequent dose of the pharmaceutical composition are the same.

Embodiment 75: The pharmaceutical composition according to embodiment 74, wherein the first dose of the pharmaceutical composition, the second dose of the pharmaceutical composition, and the subsequent dose of the pharmaceutical composition contain 100 mg hum13B8-b.

Embodiment 76: The pharmaceutical composition according to embodiment 73, wherein the subsequent dose of the pharmaceutical composition is administered every 12 weeks at least up to 16 weeks.

Embodiment 77: The pharmaceutical composition according to embodiment 73, wherein the subsequent dose of the pharmaceutical composition is administered every 4 weeks at least up to 28 weeks.

Embodiment 78: The pharmaceutical composition according to embodiment 73, wherein the subsequent dose of the pharmaceutical composition is administered every 12 weeks at least up to 40 weeks, at least up to 52 weeks, at least up to 72 weeks, or longer.

Embodiment 79: The pharmaceutical composition according to embodiment 71, wherein the pharmaceutical composition is administered to the patient by subcutaneous injection.

Embodiment 80: The pharmaceutical composition according to embodiment 71, wherein the patient has an affected Scalp Surface Area (SSA) of at least 50%, a Psoriasis Area and Severity Index (PASI) score of at least 16.7, and/or a Physician Global Assessment (PGA) score of 3 (“moderate”) or 4 (“severe”).

Embodiment 81: The pharmaceutical composition according to embodiment 71, wherein the patient is male, White, and/or at least 44 years of age.

Embodiment 82: The pharmaceutical composition according to embodiment 71, wherein administration of the pharmaceutical composition results in the patient achieving an Investigator Global Assessment (IGA) score for the scalp of “clear” or “almost clear” with at least a 2-point reduction from Baseline at Week 16.

Embodiment 83: The pharmaceutical composition according to embodiment 71, wherein administration of the pharmaceutical composition results in the patient achieving an Investigator Global Assessment (IGA) score for the scalp of “clear” or “almost clear” with at least a 2-point reduction from Baseline at Week 12.

Embodiment 84: The pharmaceutical composition according to embodiment 71, wherein administration of the pharmaceutical composition results in the patient achieving an Investigator Global Assessment (IGA) Scalp Response Rate of 0 (clear) or 1 (almost clear) at Week 12.

Embodiment 85: The pharmaceutical composition according to embodiment 71, wherein administration of the pharmaceutical composition results in the patient achieving Psoriasis Scalp Severity Index (PSSI) 90 at Week 16.

Embodiment 86: The pharmaceutical composition according to embodiment 71, wherein administration of the pharmaceutical composition results in the patient achieving Psoriasis Scalp Severity Index (PSSI) 90 at Week 12.

Embodiment 87: A pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) score for the scalp of “clear” or “almost clear” with at least a 2-point reduction from Baseline at Week 16.

Embodiment 88: A pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) score for the scalp of “clear” or “almost clear” with at least a 2-point reduction from Baseline at Week 12.

Embodiment 89: A pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving an Investigator Global Assessment (IGA) Scalp Response Rate of 0 (clear) or 1 (almost clear) at Week 12.

Embodiment 90: A pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the antibody results in the patient achieving Psoriasis Scalp Severity Index (PSSI) 90 at Week 16.

Embodiment 91: A pharmaceutical composition of an anti-IL-23p19 antibody hum13B8-b for the treatment of moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the pharmaceutical composition is administered to the patient on week 0, a second dose of the pharmaceutical composition is administered to the patient at 4 weeks, and a subsequent dose of the pharmaceutical composition is administered to the patient every 4 to 12 weeks thereafter; wherein the pharmaceutical composition is administered to the patient by subcutaneous injection; and wherein administration of the pharmaceutical composition results in the patient achieving Psoriasis Scalp Severity Index (PSSI) 90 at Week 12.

Embodiment 92: A method of treating psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Embodiment 93: The method according to embodiment 92, wherein the psoriasis of the scalp is moderate to severe psoriasis of the scalp.

Embodiment 94: The method according to embodiment 92, wherein a therapeutically effective amount of hum13B8-b is administered to the patient.

Embodiment 95: The method according to embodiment 92, wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter.

Embodiment 96: The method according to embodiment 95 wherein the first dose, the second dose, and the subsequent dose are the same.

Embodiment 97: The method according to embodiment 96, wherein the first dose, the second dose, and the subsequent dose are 100 mg.

Embodiment 98: The method according to embodiment 95, wherein the subsequent dose is administered every 12 weeks at least up to 16 weeks.

Embodiment 99: The method according to embodiment 95, wherein the subsequent dose is administered every 4 weeks at least up to 28 weeks.

Embodiment 100: The method according to embodiment 95, wherein the subsequent dose is administered every 12 weeks at least up to 40 weeks, at least up to 52 weeks, at least up to 72 weeks, or longer.

Embodiment 101: The method according to embodiment 92, wherein the antibody is administered to the patient by subcutaneous injection.

Embodiment 102: The method according to embodiment 92, wherein the patient has an affected Scalp Surface Area (SSA) of at least 50%, a Psoriasis Area and Severity Index (PASI) score of at least 16.7, and/or a Physician Global Assessment (PGA) score of 3 (“moderate”) or 4 (“severe”).

Embodiment 103: The method according to embodiment 92, wherein the patient is male, White, and/or at least 44 years of age.

Embodiment 104: The method according to embodiment 92, wherein administration of the antibody results in the patient achieving an Investigator Global Assessment (IGA) score for the scalp of “clear” or “almost clear” with at least a 2-point reduction from Baseline at Week 16.

Embodiment 105: The method according to embodiment 92, wherein administration of the antibody results in the patient achieving an Investigator Global Assessment (IGA) score for the scalp of “clear” or “almost clear” with at least a 2-point reduction from Baseline at Week 12.

Embodiment 106: The method according to embodiment 92, wherein administration of the antibody results in the patient achieving an Investigator Global Assessment (IGA) Scalp Response Rate of 0 (clear) or 1 (almost clear) at Week 12.

Embodiment 107: The method according to embodiment 92, wherein administration of the antibody results in the patient achieving Psoriasis Scalp Severity Index (PSSI) 90 at Week 16.

Embodiment 108: The method according to embodiment 92, wherein administration of the antibody results in the patient achieving Psoriasis Scalp Severity Index (PSSI) 90 at Week 12.

Embodiment 109: A method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) score for the scalp of “clear” or “almost clear” with at least a 2-point reduction from Baseline at Week 16.

Embodiment 110: A method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) score for the scalp of “clear” or “almost clear” with at least a 2-point reduction from Baseline at Week 12.

Embodiment 111: A method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving an Investigator Global Assessment (IGA) Scalp Response Rate of 0 (clear) or 1 (almost clear) at Week 12.

Embodiment 112: A method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving Psoriasis Scalp Severity Index (PSSI) 90 at Week 16.

Embodiment 113: A method of treating moderate to severe psoriasis of the scalp comprising administering an anti-IL-23p19 antibody hum13B8-b to a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter; wherein hum13B8-b is administered to the patient by subcutaneous injection; and wherein administration of hum13B8-b results in the patient achieving Psoriasis Scalp Severity Index (PSSI) 90 at Week 12.

Embodiment 114: A pharmaceutical composition of an anti-IL-23p19 antibody hum13B8-b for the manufacture of a medicament for treating psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Embodiment 115: The pharmaceutical composition according to embodiment 114, wherein the psoriasis of the scalp is moderate to severe psoriasis of the scalp.

Embodiment 116: The pharmaceutical composition according to embodiment 114, wherein a first dose of the medicament is administered to the patient on week 0, a second dose of the medicament is administered to the patient at 4 weeks, and a subsequent dose of the medicament is administered to the patient every 4 to 12 weeks thereafter.

Embodiment 117: The pharmaceutical composition according to embodiment 116, wherein the first dose of the medicament, the second dose of the medicament, and the subsequent dose of the medicament are the same.

Embodiment 118: The pharmaceutical composition according to embodiment 117, wherein the first dose of the medicament, the second dose of the medicament, and the subsequent dose of the medicament contain 100 mg hum13B8-b.

Embodiment 119: The pharmaceutical composition according to embodiment 116, wherein the subsequent dose of the medicament is administered every 12 weeks at least up to 16 weeks.

Embodiment 120: The pharmaceutical composition according to embodiment 116, wherein the subsequent dose of the medicament is administered every 4 weeks at least up to 28 weeks.

Embodiment 121: The pharmaceutical composition according to embodiment 116, wherein the subsequent dose of the medicament is administered every 12 weeks at least up to 40 weeks, at least up to 52 weeks, at least up to 72 weeks, or longer.

Embodiment 122: The pharmaceutical composition according to embodiment 114, wherein the medicament is administered to the patient by subcutaneous injection.

Embodiment 123: The pharmaceutical composition according to embodiment 114, wherein the patient has an affected Scalp Surface Area (SSA) of at least 50%, a Psoriasis Area and Severity Index (PASI) score of at least 16.7, and/or a Physician Global Assessment (PGA) score of 3 (“moderate”) or 4 (“severe”).

Embodiment 124: The pharmaceutical composition according to embodiment 114, wherein the patient is male, White, and/or at least 44 years of age.

Embodiment 125: The pharmaceutical composition according to embodiment 114, wherein administration of the medicament results in the patient achieving an Investigator Global Assessment (IGA) score for the scalp of “clear” or “almost clear” with at least a 2-point reduction from Baseline at Week 16.

Embodiment 126: The pharmaceutical composition according to embodiment 114, wherein administration of the medicament results in the patient achieving an Investigator Global Assessment (IGA) score for the scalp of “clear” or “almost clear” with at least a 2-point reduction from Baseline at Week 12.

Embodiment 127: The pharmaceutical composition according to embodiment 114, wherein administration of the medicament results in the patient achieving an Investigator Global Assessment (IGA) Scalp Response Rate of 0 (clear) or 1 (almost clear) at Week 12.

Embodiment 128: The pharmaceutical composition according to embodiment 114, wherein administration of the medicament results in the patient achieving Psoriasis Scalp Severity Index (PSSI) 90 at Week 16.

Embodiment 129: The pharmaceutical composition according to embodiment 114, wherein administration of the medicament results in the patient achieving Psoriasis Scalp Severity Index (PSSI) 90 at Week 12.

Embodiment 130: The pharmaceutical composition of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) score for the scalp of “clear” or “almost clear” with at least a 2-point reduction from Baseline at Week 16.

Embodiment 131: The pharmaceutical composition of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) score for the scalp of “clear” or “almost clear” with at least a 2-point reduction from Baseline at Week 12.

Embodiment 132: The pharmaceutical composition of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving an Investigator Global Assessment (IGA) Scalp Response Rate of 0 (clear) or 1 (almost clear) at Week 12.

Embodiment 133: The pharmaceutical composition of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving Psoriasis Scalp Severity Index (PSSI) 90 at Week 16.

Embodiment 134: The pharmaceutical composition of an anti-IL-23p19 antibody hum13B8-b for the manufacture of a medicament for treating moderate to severe psoriasis of the scalp in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein a first dose of the medicament is administered to the patient on week 0, a second dose of the medicament is administered to the patient at 4 weeks, and a subsequent dose of the medicament is administered to the patient every 4 to 12 weeks thereafter; wherein the medicament is administered to the patient by subcutaneous injection; and wherein administration of the medicament results in the patient achieving Psoriasis Scalp Severity Index (PSSI) 90 at Week 12.

### Examples

The Examples that follow are illustrative of specific embodiments of the disclosure, and various uses thereof. They are set forth for explanatory purposes only and should not be construed as limiting the scope of the disclosure in any way.

### Example 1: Administration of Tildrakizumab in Subjects with Plaque Psoriasis of the Scalp—Phase 3 Study Proposal

This is a multicenter, randomized, double-blind, placebo-controlled study to assess the efficacy and safety of tildrakizumab in the treatment of moderate to severe psoriasis of the scalp.

Subjects with a clinical diagnosis of chronic plaque psoriasis for at least 6 months, having moderate to severe plaque psoriasis of the scalp at Screening and at Baseline (Investigator Global Assessment [IGA mod 2011 (Scalp)]) of ≥3, Psoriasis Scalp Severity Index [PSSI] score of ≥12, and 30% or higher of scalp surface area affected) as well as moderate to severe plaque psoriasis at Screening and Baseline (IGA Mod 2011 (whole body) of ≥3, Psoriasis Area and Severity Index [PASI] score≥12 and Body Surface Area [BSA] involvement of ≥10%) and who are considered candidates for systemic therapy were enrolled in the study (NCT03897088).

After a Screening Period of 28 days, all 231 subjects were randomly allocated (in a 1:1 ratio after stratification according to previous use of TNF-alpha inhibitors and Baseline body weight [≤90 kg or ≥90 kg]) on Day 1 to receive either tildrakizumab 100 mg or placebo by subcutaneous (SC) injection on Week 0 (Day 1), and on Week 4. Subjects received the first dose of study treatment within 24 hours of randomization.

For Week 16 to Week 52, subjects initially randomized to placebo were switched to tildrakizumab, receiving 100 mg at Weeks 16, 20, 32, and 44. Subjects initially randomized to tildrakizumab 100 mg continued to receive tildrakizumab at Weeks 16, 28, 40, and 52. In order to maintain the blind, subjects in both treatment arms received matching placebo injections at specified time points as described in the Schedule of Activities (SoA).

After Week 52 (or early termination of study treatment prior to Week 52), the study treatment was stopped, and the subjects entered the 20-week Observational Safety Follow-up Period following the last dose of study treatment. During the follow-up period, subjects continued on study-approved concomitant medications only, however may be placed on other appropriate therapies for safety concerns or significant worsening of psoriasis based on the judgment of the Investigator. The subjects did not receive study treatment during the follow-up period.

The subject's disease activity (response to study treatment) was evaluated using the IGA Mod 2011 (scalp), PSSI, PASI, IGA (scalp only), PGA-S (whole body), IGA Mod 2011 (whole body), Scalp Itch Numeric Rating Scale (NRS), BSA, and Dermatology Life Quality Index (DLQI) scores. Results are shown in the Examples and Tables below.

### 1. Number of Subjects:

The study randomized 231 subjects with moderate to severe psoriasis of the scalp and treated one arm with tildrakizumab 100 mg (n=117) and the other with placebo (n=114).

### 2. Treatment Groups and Duration:

The study duration per subject was approximately 18 to 19 months and the total duration of the study will be approximately 2.5 years.

The study consisted of a Screening Period of 28 days, a 52-week treatment period (16-week double-blind placebo-controlled treatment period followed by a 36-week double-blind active treatment period), and a 20-week Observational Safety Follow-up Period.

Part 1 (Day 1 to Week 16): Double-blind placebo-controlled, where subjects received either tildrakizumab 100 mg (Arm A; n=117) or placebo (Arm B; n=114) by SC injection at Weeks 0, and 4. The treatment period for the double-blind Part 1 of the study is 16 weeks, and assessments of efficacy and safety were performed at Week 16

Part 2 (Week 16 to Week 52): At Week 16, subjects initially randomized to placebo were switched over to receive tildrakizumab 100 mg at Weeks 16, 20, 32, and 44. Subjects initially randomized to tildrakizumab 100 mg continued to receive active drug at Weeks 16, 28, 40, and 52. In order to maintain the blind, subjects in both treatment arms received matching placebo injections at specified time points. Assessments of efficacy and safety were performed after the date of injection at Week 16 and before or on the date of injection at Week 52 (or the 1st injection before Week 52) plus 28 days.

Part 3 (Week 52 to Week 72): After Week 52 (or early termination of study treatment prior to Week 52), subjects entered the 20-week Observational Safety Follow-up Period where tildrakizumab treatment was stopped. Assessments of safety after the date of injection at Week 52 (or the last injection, or early termination of Part 1 and Part 2 prior to Week 52) were performed for 20 weeks.

### 3. Statistical Methods:

The primary efficacy endpoint for this study was the proportion of subjects with IGA mod 2011(scalp) score of “clear” and “almost clear” with at least 2-point reduction from baseline at Week 16. The key secondary efficacy endpoints were the proportion of subjects with at least 90% improvement in the PSSI (PSSI90) from baseline at Week 16, the proportion of subjects with IGA (scalp only) score of “clear” and “almost clear” with at least 2-point reduction from baseline at Week 16, the proportion of subjects with IGA mod 2011(scalp) score of “clear” and “almost clear” with at least 2-point reduction from baseline at Week 12 and the proportion of subjects with at least 90% improvement in the PSSI (PSSI90) from baseline at Week 12. For each endpoint, the tildrakizumab 100 mg dose was compared with placebo. Both the primary and key secondary efficacy endpoints were analyzed with Cochran-Mantel-Haenszel test stratified by body weight (≤90 kg or >90 kg) and prior exposure to TNF-alpha inhibitors for psoriasis (Yes/No) using the Intent-to-treat (ITT) population. All analyses were conducted for Per Protocol Set population as supportive. In addition, the Mantel-Haenszel common risk (the response rate) difference between tildrakizumab 100 mg dose arms and placebo and the 95% confidence interval were estimated. A mixed model repeated measures (MMRM) procedure approach was also applied for all time point data up to Week 16.

TABLE 1
Objectives and Endpoints.

| Objectives | Endpoints |
|---|---|
| Primary Efficacy Objective | Primary Efficacy Endpoint |
| To assess the efficacy of tildrakizumab in | The proportion of subjects with IGA mod |
| subjects with moderate to severe scalp | 2011 (scalp) score of “clear” and “almost |
| psoriasis, as measured by the proportion of | clear” with at least 2-point reduction from |
| subjects who achieve Investigator Global | Baseline at Week 16 |
| Assessment (IGA) mod 2011 (scalp) score | |
| of “clear” and “almost clear” with at least | |
| 2-point reduction from Baseline at Week 16. | |
| Primary Safety Objective | Primary Safety Endpoint |
| To assess the safety and tolerability of | The percentage of subjects with incidence, |
| tildrakizumab in subjects with moderate to | seriousness and severity of all adverse |
| severe plaque psoriasis of the scalp over | events. |
| 52 weeks. | The percentage of subjects with severe |
| | infections, defined as any infection meeting |
| | the regulatory definition of serious adverse |
| | event, or any infection requiring intravenous |
| | antibiotics whether or not reported as a |
| | serious event as per the regulatory |
| | definition. |
| | The percentage of subjects with |
| | malignancies (excluding carcinoma in situ |
| | of the cervix). |
| | The percentage of subjects with |
| | non-melanoma skin cancer. |
| | The percentage of subjects with melanoma |
| | skin cancer. |
| | The percentage of subjects with Major |
| | Adverse Cardiovascular Events. |
| | The percentage of subjects with study |
| | treatment-related hypersensitivity reactions |
| | (eg, anaphylaxis, urticaria, angioedema, |
| | etc.). |
| | The percentage of subjects with injection |
| | site reactions (eg, pain, erythema, edema |
| | etc). |
| Secondary Objectives | Secondary Endpoints |
| To assess the efficacy of tildrakizumab in | The proportions of subjects with at least 90% |
| subjects with moderate to severe plaque | improvement from Baseline in the PSSI at |
| psoriasis of the scalp, as measured by the | Week 16. (Key secondary endpoint). |
| proportion of subjects who achieve at least | The proportion of subjects with IGA (scalp |
| 90% improvement from Baseline in the | only) score of “clear” and “almost clear” |
| Psoriasis Scalp Severity Index (PSSI) | with at least 2-point reduction from |
| response at Week 16 compared with placebo. | Baseline at Week 16. (Key secondary endpoint). |
| To assess the efficacy of tildrakizumab in | The proportion of subjects with IGA mod |
| subjects with moderate to severe scalp | 2011 (scalp) score of “clear” and “almost |
| psoriasis, as measured by the proportion of | clear” with at least 2-point reduction from |
| subjects who achieve IGA (Scalp only) of | Baseline at Week 12 (key secondary |
| “clear” and “almost clear” with at least | endpoint) |
| 2-point reduction from Baseline at Week 16. | The proportion of subjects with at least 90% |
| To assess the efficacy of tildrakizumab in | improvement from Baseline in the PSSI at |
| subjects with moderate to severe scalp | Week 12. (Key secondary endpoint). |
| psoriasis, as measured by the proportion of | Mean percentage change in PSSI score from |
| subjects who achieve Investigator Global | Baseline to Week 16. |
| Assessment (IGA) mod 2011 (scalp) score | The proportion of subjects achieving |
| of “clear” and “almost clear” with at least | PSSI 75 at Week 16. |
| 2-point reduction from Baseline at Week 12. | The proportion of subjects achieving |
| To assess the efficacy of tildrakizumab in | PSSI 100 at Week 16. |
| subjects with moderate to severe plaque | Mean percentage change in scalp surface |
| psoriasis of the scalp, as measured by the | area involvement from Baseline to |
| proportion of subjects who achieve at least | Week 16. |
| 90% improvement from Baseline in the | |
| Psoriasis Scalp Severity Index (PSSI) | |
| response at Week 12 compared with | |
| placebo. To assess the efficacy of | |
| tildrakizumab in the treatment of moderate | |
| to severe plaque psoriasis of the scalp | |
| compared with placebo as measured by | |
| scalp surface area involvement at Week 16. | |
| To assess the effect of Tildrakizumab on | Change from baseline in IGA mod 2011 |
| IGA mod 2011 (scalp) and PSSI at other | (scalp) and PSSI at other measured time |
| measured time points through week 52. | points through Week 52. |
| To assess the effect of tildrakizumab on, | Change from Baseline in IGA (scalp only), |
| IGA (scalp only), Scalp Itch NRS, PASI, | Scalp Itch NRS, PASI, PGA-S (whole |
| PGA-S (whole body), IGA mod 2011 | body), and IGA mod 2011 (whole body) at |
| (whole body) at week 12 and other | week 12 and other measured time points |
| measured time points through Week 52. | through Week 52. |
| To assess the time to response. | Time to 75% reduction in PSSI during the |
| | 16-week placebo-controlled treatment |
| | period. |
| | Time to IGA mod 2011 (scalp) response |
| | during the 16-week placebo-controlled |
| | treatment period. |
| To assess the efficacy of tildrakizumab in | The proportion of subjects achieving a 4- |
| the improvement of scalp itch as measured | point reduction in Scalp Itch NRS score |
| by the proportion of subjects achieving a 4- | from Baseline to Week 16. |
| point reduction in Scalp Itch Numeric | |
| Rating Scale (NRS) score from Baseline at | |
| Week 16 compared with placebo. | |
| To assess the efficacy of tildrakizumab in | The proportion of subjects achieving |
| the treatment of moderate to severe psoriasis | PASI 75, PASI 90, and PASI 100 at |
| (entire body including scalp) compared with | Week 16. |
| placebo as measured by Psoriasis Area and | The proportion of subjects with IGA mod |
| Severity Index (PASI), IGA mod | 2011 score (whole body) and PGA-S |
| 2011(whole body), Physician Global | (whole body) score of “clear” or “almost |
| Assessment for skin (PGA-S) score (whole | clear” with at least a 2-point reduction from |
| body), and total body surface area (BSA) | Baseline to Week 16. |
| involvement at Week 16. | Mean percentage change in total BSA |
| | involvement from Baseline to Week 16. |
| Exploratory Objectives | Exploratory Endpoints |
| To assess the effect of tildrakizumab on | Change from Baseline in DLQI score (total |
| Quality of Life measured by: | and 6 domain scores) at measured time |
| Dermatology Life Quality Index | points through Week 52. |
| (DLQI). | |

TABLE 2
Sequences

| SEQ | | |
|---|---|---|
| ID | | |
| NO: | Sequence | Description |
| 1 | Asp Ile Gln Met Thr Gln Ser Pro | hum13B8-b |
| | Ser Ser Leu Ser Ala Ser Val Gly | light |
| | Asp Arg Val Thr Ile Thr Cys Arg | chain |
| | Thr Ser Glu Asn Ile Tyr Ser Tyr | |
| | Leu Ala Trp Tyr Gln Gln Lys Pro | |
| | Gly Lys Ala Pro Lys Leu Leu Ile | |
| | Tyr Asn Ala Lys Thr Leu Ala Glu | |
| | Gly Val Pro Ser Arg Phe Ser Gly | |
| | Ser Gly Ser Gly Thr Asp Phe Thr | |
| | Leu Thr Ile Ser Ser Leu Gln Pro | |
| | Glu Asp Phe Ala Thr Tyr Tyr Cys | |
| | Gln His His Tyr Gly Ile Pro Phe | |
| | Thr Phe Gly Gln Gly Thr Lys Val | |
| | Glu Ile Lys Arg Thr Val Ala Ala | |
| | Pro Ser Val Phe Ile Phe Pro Pro | |
| | Ser Asp Glu Gln Leu Lys Ser Gly | |
| | Thr Ala Ser Val Val Cys Leu Leu | |
| | Asn Asn Phe Tyr Pro Arg Glu Ala | |
| | Lys Val Gln Trp Lys Val Asp Asn | |
| | Ala Leu Gln Ser Gly Asn Ser Gln | |
| | Glu Ser Val Thr Glu Gln Asp Ser | |
| | Lys Asp Ser Thr Tyr Ser Leu Ser | |
| | Ser Thr Leu Thr Leu Ser Lys Ala | |
| | Asp Tyr Glu Lys His Lys Val Tyr | |
| | Ala Cys Glu Val thr His Gln Gly | |
| | Leu Ser Ser Pro Val Thr Lys Ser | |
| | Phe Asn Arg Gly Glu Cys | |
| 2 | Gln Val Gln Leu Val Gln Ser Gly | hum13B8-b |
| | Ala Glu Val Lys Lys Pro Gly Ala | heavy |
| | Ser Val Lys Val Ser Cys Lys Ala | chain |
| | Ser Gly Tyr Ile Phe Ile Thr Tyr | |
| | Trp Met Thr Trp Val Arg Gln Ala | |
| | Pro Gly Gln Gly Leu Glu Trp Met | |
| | Gly Gln Ile Phe Pro Ala Ser Gly | |
| | Ser Ala Asp Tyr Asn Glu Lys Phe | |
| | Glu Gly Arg Val Thr Met Thr Thr | |
| | Asp Thr Ser Thr Ser Thr Ala Tyr | |
| | Met Glu Leu Arg Ser Leu Arg Ser | |
| | Asp Asp Thr Ala Val Tyr Tyr Cys | |
| | Ala Arg Gly Gly Gly Gly Phe Ala | |
| | Tyr Trp Gly Gln Gly Thr Leu Val | |
| | Thr Val Ser Ser Ala Ser Thr Lys | |
| | Gly Pro Ser Val Phe Pro Leu Ala | |
| | Pro Ser Ser Lys Ser Thr Ser Gly | |
| | Gly Thr Ala Ala Leu Gly Cys Leu | |
| | Val Lys Asp Tyr Phe Pro Glu Pro | |
| | Val Thr Val Ser Trp Asn Ser Gly | |
| | Ala Leu Thr Ser Gly Val His Thr | |
| | Phe Pro Ala Val Leu Gln Ser Ser | |
| | Gly Leu Tyr Ser Leu Ser Ser Val | |
| | Val Thr Val Pro Ser Ser Ser Leu | |
| | Gly Thr Gln Thr Tyr Ile Cys Asn | |
| | Val Asn His Lys Pro Ser Asn Thr | |
| | Lys Val Asp Lys Lys Val Glu Pro | |
| | Lys Ser Cys Asp Lys Thr His Thr | |
| | Cys Pro Pro Cys Pro Ala Pro Glu | |
| | Leu Leu Gly Gly Pro Ser Val Phe | |
| | Leu Phe Pro Pro Lys Pro Lys Asp | |
| | Thr Leu Met Ile Ser Arg Thr Pro | |
| | Glu Val Thr Cys Val Val Val Asp | |
| | Val Ser His Glu Asp Pro Glu Val | |
| | Lys Phe Asn Trp Tyr Val Asp Gly | |
| | Val Glu Val His Asn Ala Lys Thr | |
| | Lys Pro Arg Glu Glu Gln Tyr Asn | |
| | Ser Thr Tyr Arg Val Val Ser Val | |
| | Leu Thr Val Leu His Gln Asp Trp | |
| | Leu Asn Gly Lys Glu Tyr Lys Cys | |
| | Lys Val Ser Asn Lys Ala Leu Pro | |
| | Ala Pro Ile Glu Lys Thr Ile Ser | |
| | Lys Ala Lys Gly Gln Pro Arg Glu | |
| | Pro Gln Val Tyr Thr Leu Pro Pro | |
| | Ser Arg Asp Glu Leu Thr Lys Asn | |
| | Gln Val Ser Leu Thr Cys Leu Val | |
| | Lys Gly Phe Tyr Pro Ser Asp Ile | |
| | Ala Val Glu Trp Glu Ser Asn Gly | |
| | Gln Pro Glu Asn Asn Tyr Lys Thr | |
| | Thr Pro Pro Val Leu Asp Ser Asp | |
| | Gly Ser Phe Phe Leu Tyr Ser Lys | |
| | Leu Thr Val Asp Lys Ser Arg Trp | |
| | Gln Gln Gly Asn Val Phe Ser Cys | |
| | Ser Val Met His Glu Ala Leu His | |
| | Asn His Tyr Thr Gln Lys Ser Leu | |
| | Ser Leu Ser Pro Gly Lys | |
| 3 | Gly Tyr Ile Phe Ile Thr Tyr Trp | HC CDR1 |
| | Met Thr | |
| 4 | Gln Ile Phe Pro Ala Ser Gly Ser | HC CDR2 |
| | Ala Asp Tyr Asn Glu Lys Phe Glu | |
| 5 | Gly Gly Gly Gly Phe Ala Tyr | HC CDR3 |
| 6 | Arg Thr Ser Glu Asn Ile Tyr Ser | LC CDR1 |
| | Tyr Leu Ala | |
| 7 | Asn Ala Lys Thr Leu Ala Glu | LC CDR2 |
| 8 | Gln His His Tyr Gly Ile Pro Phe | LC CDR3 |
| | Thr | |

### Example 2: Results of the Administration of Tildrakizumab in Subjects with Plaque Psoriasis of the Scalp: Part 1

Following completion of part 1 of the 16-week Phase 3 clinical trial protocol described in Example 1, the following results were obtained. In part 1, the protocol was double-blinded, placebo-controlled, and subjects received either tildrakizumab 100 mg (Arm A; n=117) or placebo (Arm B; n=114) SC at Weeks 0 and 4.

The group labeled Modified Intent To Treat (mITT) include all subjects randomized under protocol amendments 01, 02, or 03 who were dispensed study treatment. The group labeled Uniform Intent To Treat (uITT) include all subjects randomized under protocol amendments 02 or 03 who were dispensed study treatment.

Table 3 provides a summary of the disposition of subjects for part 1 of the study and includes all randomized subjects who have been dispensed study treatment.

Tables 4 (mITT) and 5 (uITT) provide summaries of the demographic and baseline characteristics for the subjects in the mITT and uITT groups in part 1 of the study.

Table 6 provides an overall summary of treatment-emergent adverse events for part 1 of the study.

Table 7 provides a summary of subjects with treatment-emergent adverse events by system organ class and Table 8 provide a summary of the subjects with treatment-emergent adverse events by system organ class and relationship to study drug for part 1.

Table 9 provides a summary of the extent of exposure for part 1, as baseline (week 0) and week 4.

Tables 10 (mITT group) and 11 (uITT group) provide an analysis of the primary efficacy outcome, the IGA Mod 2011 (Scalp) response rate, at Week 16, for part 1. Table 12 provides an analysis of the primary efficacy outcome, the IGA Mod 2011 (Scalp) response rate, at Week 16, for part 1 for the Per Protocol (PP) group. The PP group includes all subjects in the mITT population who had completed the 16-week placebo-controlled treatment without any major protocol deviation that could impact data interpretability for the primary efficacy endpoint.

Tables 13 (ITT group) and 14 (mITT group) provide an analysis of the key secondary efficacy outcome, the PSSI-90 response rate, at Week 16, for part 1.

Tables 15 (ITT group) and 16 (mITT group) provide an analysis of a further secondary efficacy outcome, the IGA (Scalp) response rate, at Week 16, for part 1.

Table 17 (mITT group) provides an analysis of the key secondary efficacy outcome, the IGA Mod 2011 (Scalp) response rate, at Week 12, for part 1.

When taken together, Tables 3-17 demonstrate the successful completion of part 1 of the Phase 3 clinical trial protocol described in Example 1 and provide the results of treatment of plaque psoriasis of the scalp with tildrakizumab over 16 weeks. Subjects treated with tildrakizumab showed a significant improvement in plaque psoriasis of the scalp as measured by IGA Mod 2011 (Scalp), PSSI-90, and the IGA (scalp). Furthermore, the treatment was well tolerated, with no subjects experiencing a drug-related serious treatment emergent adverse event (TEAE).

### Example 3: Results of the Administration of Tildrakizumab in Subjects with Plaque Psoriasis of the Scalp: Part 2

The multicenter, randomized, double-blind, placebo-controlled trial (NCT03897088) comprised 231 subjects with moderate to severe psoriasis of the scalp who were treated with tildrakizumab 100 mg (n=117) or placebo (n=114) at Week 0 and 4 and every 12 weeks thereafter. Of the 231 randomized subjects, 217 subjects completed Part 1 (Day 1 to Week 16). For Week 16 to Week 52, subjects randomized to placebo were switched to tildrakizumab receiving 100 mg at Weeks 16, 20, 32, and 44.

The primary endpoint was the proportion of subjects with IGA score for the scalp of “clear” and “almost clear” with at least 2-point reduction from Baseline at Week 16.

Other evaluated outcomes included the proportion of subjects achieving a) Psoriasis Scalp Severity Index (PSSI) 90 (≥90% improvement from Baseline in PSSI) at Week 16; b) subjects achieving PSSI 90 at Week 12; and c) IGA for the scalp score of “clear” or “almost clear” with at a least a 2-point reduction from Baseline at Week 12.

Subjects in the tildrakizumab and placebo treatment groups were predominantly men (60.2%) and white (78.9), with a mean age of 44.8 years. At baseline, these subjects had a median affected scalp surface area (SSA) of 50%, a median PASI score of 16.7, and PGA score of 3 (“moderate”) or 4 (“severe”) in 87.1% and 12.3%, respectively.

Table 18 (mITT group) shows the outcome by IGA Mod 2011 (Scalp) response rate by visit over the 52 weeks for Part 2.

Table 19 (ITT group) shows the outcome by PSSI 90 response rate by visit over the 52 weeks for Part 2.

Table 20 shows the proportion of subjects with investigator global assessment mod 2011 (scalp) score of “clear” and “almost clear” with at least 2-point reduction from baseline at week 16.

Table 21 shows the proportion of subjects with at least 90% improvement from baseline in the psoriasis scalp severity index at week 16.

Table 22 shows the mean percentage change in psoriasis scalp severity index score from baseline to week 16.

Table 23 shows the proportion of subjects achieving psoriasis scalp severity index 75 at week 16.

Table 24 shows the proportion of subjects achieving psoriasis scalp severity index 100 at week 16.

Table 25 shows the mean percentage change in scalp surface area (SSA) involvement from baseline to week 16.

Table 26 shows the time to 75% reduction in psoriasis scalp severity index during 16-week placebo-controlled treatment period.

Table 27 shows the time to investigator global assessment mod 2011 (Scalp) response during the 16-week placebo-controlled treatment period.

Table 28 shows the proportion of subjects achieving a 4-point reduction in itch numeric rating scale score from baseline to week 16.

Table 29 shows the proportion of subjects achieving psoriasis area and severity index (PASO 75, psoriasis area and severity index 90, and psoriasis area and severity index 100 at week 16.

Table 30 shows the proportion of subjects with physician's global assessment score (Whole body) score of “clear” or “almost clear” with at least a 2-point reduction from baseline to week 16.

Table 31 shows the mean percentage change in total body surface area (BSA) involvement from baseline to week 16.

Table 32 shows the proportion of subjects with investigator global assessment (Scalp only) score of “clear” and “almost clear” with at least 2-point reduction from baseline at week 16.

Table 33 shows the proportion of subjects with investigator global assessment mod 2011 score (Whole body) of “clear” and “almost clear” with at least 2-point reduction from baseline at week 16.

Table 34 shows the proportion of subjects with IGA mod 2011 (Scalp) score of “clear” and “almost clear” with at least 2-point reduction from baseline.

Table 35 shows the proportion of subjects with at least 90% improvement from baseline in the psoriasis scalp severity index at week 12.

Table 36 shows change in investigator global assessment mod 2011 (Scalp) from baseline at week 52.

Table 37 shows the change in IGA mod 2011 (whole-body) from baseline at week 52.

Table 38 shows the mean change in psoriasis scalp severity index score from baseline at week 52.

Table 39 shows the change from baseline in investigator global assessment (Scalp only) at week 52.

Table 40 shows the change from baseline in scalp itch numeric rating scale (NRS) score at week 52.

Table 41 shows the mean change in PASI score from baseline at week 52.

Table 42 shows the change in physician global assessment for skin (whole body) from baseline at week 52.

Table 43 shows the change from baseline in dermatology life quality index score (total and 6 domain scores) at measured time points through week 52.

Table 44 shows a summary of the reported adverse events through week 72.

TABLE 3
Subjects Disposition

| Status | Placebo | Tildrakizumab 100 mg | Total | |
|---|---|---|---|---|
| Screened | | | 314 | |
| Screen Failure | | | 83 | (26.4) |
| Reason for | | | | |
| Screen Failure | | | | |
| Inclusion 1 | | | 1 | (0.3) |
| Inclusion 2 | | | 1 | (0.3) |
| Inclusion 3 | | | 6 | (1.9) |
| Inclusion 4 | | | 8 | (2.5) |
| Inclusion 6 | | | 5 | (1.6) |
| Inclusion 9 | | | 1 | (0.3) |
| Inclusion 10 | | | 12 | (3.8) |
| Exclusion 1 | | | 15 | (4.8) |
| Exclusion 4 | | | 1 | (0.3) |
| Exclusion 5 | | | 6 | (1.9) |
| Exclusion 6 | | | 2 | (0.6) |
| Exclusion 7 | | | 2 | (0.6) |
| Exclusion 13 | | | 7 | (2.2) |
| Exclusion 15 | | | 2 | (0.6) |
| Exclusion 16 | | | 2 | (0.6) |
| Other | | | 12 | (3.8) |

Note:
Denominator for percentages of screen failure is the number of subjects screened. All other denominator for percentages is the number of subjects randomized.
[1] All randomized subjects who have been dispensed study treatment.
[2] All subjects randomized under protocol amendments 01, 02, or 03 who were dispensed study treatment.
[3] All subjects randomized under protocol amendments 02 or 03 who were dispensed study treatment.
[4] All subjects randomized who have received at least one dose of study treatment.
[5] All subjects in the mITT population who have completed the 16-week placebo-controlled treatment without any major protocol deviation that could impact data interpretability for the primary efficacy endpoint, following the same exclusion criteria as for the PP Set.
[6] Part 1 completers had a primary or key secondary efficacy assessment at or before study day 115 or a study treatment at Week 16.
[7] Subject 14-014 did not complete Week 16 due to COVID-19 AE, but continued study participation

TABLE 4
Demographic and Baseline Characteristics mITT

| | Placebo | | Tildrakizumab 100 mg | | All Subjects | |
|---|---|---|---|---|---|---|
| Variable | (N = 82) | | (N = 89) | | (N = 171) | |
| Age (years) | | | | | | |
| n | 82 | | 89 | | 171 | |
| Mean | 45.4 | | 44.2 | | 44.8 | |
| SD | 12.93 | | 15.08 | | 14.06 | |
| Median | 46.0 | | 41.0 | | 43.0 | |
| Range | (18, 79) | | (18, 78) | | (18, 79) | |
| Sex n (%) | | | | | | |
| Male | 44 | (53.7) | 59 | (66.3) | 103 | (60.2) |
| Female | 38 | (46.3) | 30 | (33.7) | 68 | (39.8) |
| Missing | 0 | | 0 | | 0 | |
| Race n (%) | | | | | | |
| White | 64 | (78.0) | 71 | (79.8) | 135 | (78.9) |
| Asian | 7 | (8.5) | 4 | (4.5) | 11 | (6.4) |
| Black or African American | 5 | (6.1) | 9 | (10.1) | 14 | (8.2) |
| American Indian or Alaska | 1 | (1.2) | 2 | (2.2) | 3 | (1.8) |
| Native | | | | | | |
| Native Hawaiian or Other | 2 | (2.4) | 3 | (3.4) | 5 | (2.9) |
| Pacific Islander | | | | | | |
| Other | 3 | (3.7) | 0 | | 3 | (1.8) |
| Missing | 0 | | 0 | | 0 | |
| Ethnicity n (%) | | | | | | |
| Hispanic or Latino | 29 | (35.4) | 31 | (34.8) | 60 | (35.1) |
| Not Hispanic or Latino | 53 | (64.6) | 58 | (65.2) | 111 | (64.9) |
| Missing | 0 | | 0 | | 0 | |
| Height (cm) | | | | | | |
| n | 82 | | 89 | | 171 | |
| Mean | 169.34 | | 170.23 | | 169.80 | |
| SD | 10.184 | | 8.857 | | 9.499 | |
| Median | 168.00 | | 171.00 | | 170.00 | |
| Range | (147.0, 199.0) | | (145.0, 186.0) | | (145.0, 199.0) | |
| Weight (kg) | | | | | | |
| n | 82 | | 89 | | 171 | |
| Mean | 90.05 | | 88.33 | | 89.15 | |
| SD | 21.682 | | 20.898 | | 21.232 | |
| Median | 87.80 | | 88.50 | | 88.00 | |
| Range | (50.0, 149.7) | | (46.8, 136.0) | | (46.8, 149.7) | |
| Weight Category n (%) | | | | | | |
| <=90 kg | 44 | (53.7) | 49 | (55.1) | 93 | (54.4) |
| >90 kg | 38 | (46.3) | 40 | (44.9) | 78 | (45.6) |
| Missing | 0 | | 0 | | 0 | |
| BMI (kg/m²) | | | | | | |
| n | 82 | | 89 | | 171 | |
| Mean | 31.35 | | 30.34 | | 30.82 | |
| SD | 6.928 | | 6.173 | | 6.546 | |
| Median | 29.30 | | 29.90 | | 29.50 | |
| Range | (19.2, 52.3) | | (15.2, 47.1) | | (15.2, 52.3) | |
| Prior Use of TNF-alpha | | | | | | |
| Inhibitors n (%) | | | | | | |
| Yes | 7 | (8.5) | 9 | (10.1) | 16 | (9.4) |
| No | 75 | (91.5) | 80 | (89.9) | 155 | (90.6) |
| Missing | 0 | | 0 | | 0 | |
| Protocol version at | | | | | | |
| Enrollment n (%) | | | | | | |
| Original Protocol | 0 | | 0 | | 0 | |
| Protocol Amendment 01 | 37 | (45.1) | 45 | (50.6) | 82 | (48.0) |
| Protocol Amendments 02, 03 | 45 | (54.9) | 44 | (49.4) | 89 | (52.0) |
| Missing | 0 | | 0 | | 0 | |
| Baseline IGA (Scalp Only) | | | | | | |
| n (%) | | | | | | |
| 3 = Moderate | 69 | (84.1) | 77 | (86.5) | 146 | (85.4) |
| 4 = Severe | 12 | (14.6) | 12 | (13.5) | 24 | (14.0) |
| Missing | 1 | (1.2) | 0 | | 1 | (0.6) |
| Baseline PASI | | | | | | |
| n | 82 | | 89 | | 171 | |
| Mean | 18.31 | | 19.31 | | 18.83 | |
| SD | 7.896 | | 6.725 | | 7.305 | |
| Median | 15.55 | | 17.40 | | 16.70 | |
| Range | (12.0, 55.0) | | (12.0, 39.0) | | (12.0, 55.0) | |
| Baseline PSSI | | | | | | |
| n | 82 | | 89 | | 171 | |
| Mean | 31.7 | | 33.8 | | 32.8 | |
| SD | 15.78 | | 15.11 | | 15.42 | |
| Median | 27.0 | | 28.0 | | 27.0 | |
| Range | (12, 72) | | (12, 72) | | (12, 72) | |
| Baseline SSA Involvement | | | | | | |
| n | 82 | | 89 | | 171 | |
| Mean | 55.46 | | 58.82 | | 57.21 | |
| SD | 22.874 | | 24.746 | | 23.856 | |
| Median | 50.00 | | 50.00 | | 50.00 | |
| Range | (30.0, 100.0) | | (30.0, 100.0) | | (30.0, 100.0) | |
| Baseline total BSA | | | | | | |
| Involvement | | | | | | |
| n | 82 | | 89 | | 171 | |
| Mean | 22.84 | | 24.33 | | 23.61 | |
| SD | 14.532 | | 13.777 | | 14.122 | |
| Median | 17.60 | | 19.00 | | 18.00 | |
| Range | (10.0, 85.0) | | (10.0, 70.0) | | (10.0, 85.0) | |
| Baseline PGA-S Score | | | | | | |
| (Whole Body) n (%) | | | | | | |
| 3 = Moderate | 72 | (87.8) | 77 | (86.5) | 149 | (87.1) |
| 4 = Severe | 9 | (11.0) | 12 | (13.5) | 21 | (12.3) |
| Missing | 1 | (1.2) | 0 | | 1 | (0.6) |
| Baseline Scalp Itch NRS Score | | | | | | |
| n | 79 | | 85 | | 164 | |
| Mean | 7.2 | | 7.0 | | 7.1 | |
| SD | 2.39 | | 2.44 | | 2.41 | |
| Median | 8.0 | | 7.0 | | 7.0 | |
| Range | (0, 10) | | (0, 10) | | (0, 10) | |
| Baseline IGA Mod 2011 | | | | | | |
| (Scalp), n (%) | | | | | | |
| Overall | | | | | | |
| 3 = Moderate | 64 | (78.0) | 75 | (84.3) | 139 | (81.3) |
| 4 = Severe | 15 | (18.3) | 13 | (14.6) | 28 | (16.4) |
| Missing | 3 | (3.7) | 1 | (1.1) | 4 | (2.3) |
| Subjects Enrolled under | | | | | | |
| Protocol Amendment 01 [1] | | | | | | |
| n | 37 | | 45 | | 82 | |
| 3 = Moderate | 29 | (78.4) | 35 | (77.8) | 64 | (78.0) |
| 4 = Severe | 5 | (13.5) | 9 | (20.0) | 14 | (17.1) |
| Missing | 3 | (8.1) | 1 | (2.2) | 4 | (4.9) |
| Subjects Enrolled under | | | | | | |
| Protocol Amendments | | | | | | |
| 02 and 03 [2] | | | | | | |
| n | 45 | | 44 | | 89 | |
| 3 = Moderate | 35 | (77.8) | 40 | (90.9) | 75 | (84.3) |
| 4 = Severe | 10 | (22.2) | 4 | (9.1) | 14 | (15.7) |
| Missing | 0 | | 0 | | 0 | |
| Baseline IGA Mod 2011 | | | | | | |
| (Whole Body), n (%) | | | | | | |
| 3 = Moderate | 69 | (84.1) | 77 | (86.5) | 146 | (85.4) |
| 4 = Severe | 10 | (12.2) | 11 | (12.4) | 21 | (12.3) |
| Missing | 3 | (3.7) | 1 | (.1) | 4 | (2.3) |

Note:
IGA = Investigator Global Assessment; PASI = Psoriasis Area and Severity Index; PSSI = Psoriasis Scalp Severity Index; SSA = Scalp Surface Area; BSA = Involved Body Surface Area; PGA-S = Physician Global Assessment for Skin; NRS = Numeric Rating Scale.
[1] Denominator for percentages is the number of subjects enrolled under Protocol Amendment 01.
[2] Denominator for percentages is the number of subjects enrolled under Protocol Amendments 02 and 03.

TABLE 5
Demographic and Baseline Characteristics uITT

| | Placebo | | Tildrakizumab 100 mg | | All Subjects | |
|---|---|---|---|---|---|---|
| Variable | (N = 45) | | (N = 44) | | (N = 89) | |
| Age (years) | | | | | | |
| n | 45 | | 44 | | 89 | |
| Mean | 44.4 | | 44.9 | | 44.6 | |
| SD | 13.64 | | 16.05 | | 14.80 | |
| Median | 43.0 | | 40.5 | | 41.0 | |
| Range | (18, 79) | | (20, 78) | | (18, 79) | |
| Sex n (%) | | | | | | |
| Male | 31 | (68.9) | 33 | (75.0) | 64 | (71.9) |
| Female | 14 | (31.1) | 11 | (25.0) | 25 | (28.1) |
| Missing | 0 | | 0 | | 0 | |
| Race n (%) | | | | | | |
| White | 36 | (80.0) | 37 | (84.1) | 73 | (82.0) |
| Asian | 3 | (6.7) | 3 | (6.8) | 6 | (6.7) |
| Black or African American | 4 | (8.9) | 4 | (9.1) | 8 | (9.0) |
| American Indian or Alaska | 0 | | 0 | | 0 | |
| Native | | | | | | |
| Native Hawaiian or Other | 0 | | 0 | | 0 | |
| Pacific Islander | | | | | | |
| Other | 2 | (4.4) | 0 | | 2 | (2.2) |
| Missing | 0 | | 0 | | 0 | |
| Ethnicity n (%) | | | | | | |
| Hispanic or Latino | 15 | (33.3) | 19 | (43.2) | 34 | (38.2) |
| Not Hispanic or Latino | 30 | (66.7) | 25 | (56.8) | 55 | (61.8) |
| Missing | 0 | | 0 | | 0 | |
| Height (cm) | | | | | | |
| n | 45 | | 44 | | 89 | |
| Mean | 171.02 | | 170.69 | | 170.86 | |
| SD | 9.519 | | 8.235 | | 8.859 | |
| Median | 172.00 | | 170.55 | | 171.00 | |
| Range | (147.0, 193.0) | | (152.0, 186.0) | | (147.0, 193.0) | |
| Weight (kg) | | | | | | |
| n | 45 | | 44 | | 89 | |
| Mean | 89.37 | | 89.58 | | 89.47 | |
| SD | 21.506 | | 18.933 | | 20.160 | |
| Median | 90.00 | | 89.10 | | 89.50 | |
| Range | (50.0, 139.0) | | (46.8, 136.0) | | (46.8, 139.0) | |
| Weight Category n (%) | | | | | | |
| <=90 kg | 23 | (51.1) | 23 | (52.3) | 46 | (51.7) |
| >90 kg | 22 | (48.9) | 21 | (47.7) | 43 | (48.3) |
| Missing | 0 | | 0 | | 0 | |
| BMI (kg/m²) | | | | | | |
| n | 45 | | 44 | | 89 | |
| Mean | 30.54 | | 30.62 | | 30.58 | |
| SD | 7.293 | | 5.451 | | 6.412 | |
| Median | 28.00 | | 29.70 | | 29.10 | |
| Range | (19.2, 52.3) | | (17.7, 41.1) | | (17.7, 52.3) | |
| Prior Use of TNF-alpha | | | | | | |
| Inhibitors n (%) | | | | | | |
| Yes | 4 | (8.9) | 4 | (9.1) | 8 | (9.0) |
| No | 41 | (91.1) | 40 | (90.9) | 81 | (91.0) |
| Missing | 0 | | 0 | | 0 | |
| Protocol version at | | | | | | |
| Enrollment n (%) | | | | | | |
| Original Protocol | 0 | | 0 | | 0 | |
| Protocol Amendment 01 | 0 | | 0 | | 0 | |
| Protocol Amendments 02, 03 | 45 | (100.0) | 44 | (100.0) | 89 | (100.0) |
| Missing | 0 | | 0 | | 0 | |
| Baseline IGA (Scalp Only) | | | | | | |
| n (%) | | | | | | |
| 3 = Moderate | 37 | (82.2) | 41 | (93.2) | 78 | (87.6) |
| 4 = Severe | 8 | (17.8) | 3 | (6.8) | 11 | (12.4) |
| Missing | 0 | | 0 | | 0 | |
| Baseline PASI | | | | | | |
| n | 45 | | 44 | | 89 | |
| Mean | 18.21 | | 17.70 | | 17.96 | |
| SD | 7.734 | | 5.449 | | 6.669 | |
| Median | 15.20 | | 16.30 | | 16.10 | |
| Range | (12.0, 45.7) | | (12.0, 39.0) | | (12.0, 45.7) | |
| Baseline PSSI | | | | | | |
| n | 45 | | 44 | | 89 | |
| Mean | 30.4 | | 29.6 | | 30.0 | |
| SD | 16.85 | | 12.29 | | 14.69 | |
| Median | 24.0 | | 27.0 | | 27.0 | |
| Range | (12, 72) | | (12, 66) | | (12, 72) | |
| Baseline SSA Involvement | | | | | | |
| n | 45 | | 44 | | 89 | |
| Mean | 54.56 | | 57.09 | | 55.81 | |
| SD | 22.983 | | 23.631 | | 23.208 | |
| Median | 45.00 | | 50.00 | | 50.00 | |
| Range | (30.0, 100.0) | | (30.0, 100.0) | | (30.0, 100.0) | |
| Baseline total BSA Involvement | | | | | | |
| n | 45 | | 44 | | 89 | |
| Mean | 23.12 | | 21.07 | | 22.10 | |
| SD | 15.445 | | 12.162 | | 13.878 | |
| Median | 17.00 | | 17.00 | | 17.00 | |
| Range | (10.0, 85.0) | | (10.0, 70.0) | | (10.0, 85.0) | |
| Baseline PGA-S Score | | | | | | |
| (Whole Body) n (%) | | | | | | |
| 3 = Moderate | 39 | (86.7) | 38 | (86.4) | 77 | (86.5) |
| 4 = Severe | 6 | (13.3) | 6 | (13.6) | 12 | (13.5) |
| Missing | 0 | | 0 | | 0 | |
| Baseline Scalp Itch NRS Score | | | | | | |
| n | 43 | | 40 | | 83 | |
| Mean | 7.5 | | 6.6 | | 7.1 | |
| SD | 2.21 | | 2.46 | | 2.36 | |
| Median | 8.0 | | 7.0 | | 7.0 | |
| Range | (2, 10) | | (0, 10) | | (0, 10) | |
| Baseline IGA Mod 2011 | | | | | | |
| (Scalp), n (%) | | | | | | |
| Overall | | | | | | |
| 3 = Moderate | 35 | (77.8) | 40 | (90.9) | 75 | (84.3) |
| 4 = Severe | 10 | (22.2) | 4 | (9.1) | 14 | (15.7) |
| Missing | 0 | | 0 | | 0 | |
| Subjects Enrolled under | | | | | | |
| Protocol Amendment 01 [1] | | | | | | |
| n | 0 | | 0 | | 0 | |
| 3 = Moderate | 0 | | 0 | | 0 | |
| 4 = Severe | 0 | | 0 | | 0 | |
| Missing | 0 | | 0 | | 0 | |
| Subjects Enrolled under | | | | | | |
| Protocol Amendments | | | | | | |
| 02 and 03 [2] | | | | | | |
| n | 45 | | 44 | | 89 | |
| 3 = Moderate | 35 | (77.8) | 40 | (90.9) | 75 | (84.3) |
| 4 = Severe | 10 | (22.2) | 4 | (9.1) | 14 | (15.7) |
| Missing | 0 | | 0 | | 0 | |
| Baseline IGA Mod 2011 | | | | | | |
| (Whole Body), n (%) | | | | | | |
| 3 = Moderate | 38 | (84.4) | 39 | (88.6) | 77 | (86.5) |
| 4 = Severe | 7 | (15.6) | 5 | (11.4) | 12 | (13.5) |

| | | | |
|---|---|---|---|
| Missing | 0 | 0 | 0 |

Note:
IGA = Investigator Global Assessment; PASI = Psoriasis Area and Severity Index; PSSI = Psoriasis Scalp Severity Index; SSA = Scalp Surface Area; BSA = Involved Body Surface Area; PGA-S = Physician Global Assessment for Skin; NRS = Numeric Rating Scale.
[1] Denominator for percentages is the number of subjects enrolled under Protocol Amendment 01.
[2] Denominator for percentages is the number of subjects enrolled under Protocol Amendments 02 and 03.

TABLE 6
Overall Summary of Treatment-Emergent Adverse
Events, Part 1 Safety Analysis Set

| | Placebo | Tildrakizumab 100 mg | All Subjects |
|---|---|---|---|
| | (N = 114) | (N = 117) | (N = 231) |
| Subjects with any TEAE [1] | 24 (21.1) | 41 (35.0) | 65 (28.1) |
| Subjects with any Drug-Related TEAE | 7 (6.1) | 4 (3.4) | 11 (4.8) |
| Subjects with any TEAE with an | 0 | 0 | 0 |
| Outcome of Death | | | |
| Subjects with any Serious TEAE | 0 | 1 (0.9) | 1 (0.4) |
| Subjects with any Drug-Related | 0 | 0 | 0 |
| Serious TEAE | | | |
| Subjects with any TEAE leading to | 0 | 0 | 0 |
| Discontinuation of Study Medication | | | |
| Subjects with any Drug-Related TEAE | 0 | 0 | 0 |
| Leading to Discontinuation of Study | | | |
| Medication | | | |
| Subjects with any AEs of Special | 2 (1.8) | 3 (2.6) | 5 (2.2) |
| Interest (AESI) [2] | | | |
| Injection Site Reactions | 1 (0.9) | 0 | 1 (0.4) |
| Severe Infections | 0 | 0 | 0 |
| Malignancies (Excluding | 0 | 0 | 0 |
| Carcinoma in Situ of the Cervix) | | | |
| Non-Melanoma Skin Cancer | 0 | 2 (1.7) | 2 (0.9) |
| Melanoma Skin Cancer | 0 | 0 | 0 |
| Major Adverse Cardiovascular | 0 | 0 | 0 |
| Events (MACE) | | | |
| Study Treatment-related | 1 (0.9) | 1 (0.9) | 2 (0.9) |
| Hypersensitivity Reactions | | | |
| Subjects with any Event of Clinical | 0 | 0 | 0 |
| Interest (ECI) | | | |
| Overdose of Study Treatment | 0 | 0 | 0 |
| Elevated AST or ALT Laboratory | 0 | 0 | 0 |
| Value | | | |
| Infections Require IV or | 0 | 0 | 0 |
| Intramuscular Antibiotics | | | |
| Depression, Suicidal Ideation | 0 | 0 | 0 |
| and Other Behavior Events | | | |
| Subjects with any COVID-19 Related | 0 | 2 (1.7) | 2 (0.9) |
| TEAE | | | |

Note:
Part 1 (Day 1 to Week 16) includes all AEs that first occurred or worsened in severity after the first dose of study medication, but did not start after the Week 16 study medication administration.
[1] A Treatment-emergent adverse event (TEAE) is defined as an AE that first occurred or worsened in severity after the first administration of study treatment.
[2] Treatment-emergent AESI are identified via site and sponsor blinded data review.

TABLE 7
Subjects with Treatment-Emergent Adverse Events by System
Organ Class and Preferred Term, Part 1 Safety Analysis Set

| System Organ Class/ | Placebo | Tildrakizumab 100 mg | All Subjects |
|---|---|---|---|
| Preferred Term [1] | (N = 114) | (N = 117) | (N = 231) |
| Subjects with any TEAE [2] | 24 (21.1) | 41 (35.0) | 65 (28.1) |
| Infections and infestations | 6 (5.3) | 14 (12.0) | 20 (8.7) |
| Nasopharyngitis | 1 (0.9) | 3 (2.6) | 4 (1.7) |
| Viral upper respiratory tract | 1 (0.9) | 3 (2.6) | 4 (1.7) |
| infection | | | |
| Bronchitis | 0 | 2 (1.7) | 2 (0.9) |
| Sinusitis | 0 | 2 (1.7) | 2 (0.9) |
| Upper respiratory tract | 1 (0.9) | 1 (0.9) | 2 (0.9) |
| infection | | | |
| Breast abscess | 0 | 1 (0.9) | 1 (0.4 |
| COVID-19 | 0 | 1 (0.9) | 1 (0.4) |
| Cellulitis | 0 | 1 (0.9) | 1 (0.4) |
| Hordeolum | 0 | 1 (0.9) | 1 (0.4) |
| Influenza | 1 (0.9) | 0 | 1 (0.4) |
| Skin infection | 0 | 1 (0.9) | 1 (0.4) |
| Tooth infection | 1 (0.9) | 0 | 1 (0.4) |
| Urinary tract infection | 1 (0.9) | 0 | 1 (0.4) |
| Vulvovaginal candidiasis | 1 (0.9) | 0 | 1 (0.4) |
| Skin and subcutaneous tissue | 2 (1.8) | 10 (8.5) | 12 (5.2) |
| disorders | | | |
| Pruritus | 2 (1.8) | 1 (0.9) | 3 (1.3) |
| Seborrhoeic dermatitis | 0 | 2 (1.7) | 2 (0.9) |
| Acne | 0 | 1 (0.9) | 1 (0.4) |
| Guttate psoriasis | 0 | 1 (0.9) | 1 (0.4) |
| Hypertrichosis | 0 | 1 (0.9) | 1 (0.4) |
| Post inflammatory | 0 | 1 (0.9) | 1 (0.4) |
| pigmentation change | | | |
| Rash | 0 | 1 (0.9) | 1 (0.4) |
| Rash pruritic | 0 | 1 (0.9) | 1 (0.4) |
| Urticaria | 0 | 1 (0.9) | 1 (0.4) |
| Gastrointestinal disorders | 5 (4.4) | 4 (3.4) | 9 (3.9) |
| Diarrhoea | 3 (2.6) | 2 (1.7) | 5 (2.2) |
| Constipation | 1 (0.9) | 0 | 1 (0.4) |
| Gastrooesophageal reflux | 0 | 1 (0.9) | 1 (0.4) |
| disease | | | |
| Intra-abdominal haematoma | 1 (0.9) | 0 | 1 (0.4) |
| Nausea | 0 | 1 (0.9) | 1 (0.4) |
| Vomiting | 1 (0.9) | 0 | 1 (0.4) |
| Investigations | 4 (3.5) | 4 (3.4) | 8 (3.5) |
| Alanine aminotransferase | 1 (0.9) | 1 (0.9) | 2 (0.9) |
| increased | | | |
| Blood triglycerides increased | 2 (1.8) | 0 | 2 (0.9) |
| Blood creatine phosphokinase | 0 | 1 (0.9) | 1 (0.4) |
| increased | | | |
| Blood glucose increased | 0 | 1 (0.9) | 1 (0.4) |
| Blood potassium increased | 1 (0.9) | 0 | 1 (0.4) |
| SARS-CoV-2 test positive | 0 | 1 (0.9) | 1 (0.4) |
| Nervous system disorders | 4 (3.5) | 3 (2.6) | 7 (3.0) |
| Headache | 4 (3.5) | 1 (0.9) | 5 (2.2) |
| Migraine | 0 | 1 (0.9) | 1 (0.4) |
| Sciatica | 0 | 1 (0.9) | 1 (0.4) |
| Musculoskeletal and connective | 3 (2.6) | 2 (1.7) | 5 (2.2) |
| tissue disorders | | | |
| Arthralgia | 1 (0.9) | 0 | 1 (0.4) |
| Intervertebral disc | 1 (0.9) | 0 | 1 (0.4) |
| protrusion | | | |
| Neck pain | 0 | 1 (0.9) | 1 (0.4) |
| Pain in extremity | 0 | 1 (0.9) | 1 (0.4) |
| Psoriatic arthropathy | 0 | 1 (0.9) | 1 (0.4) |
| Tendonitis | 1 (0.9) | 0 | 1 (0.4) |
| Injury, poisoning and procedural | 1 (0.9) | 3 (2.6) | 4 (1.7) |
| complications | | | |
| Foot fracture | 1 (0.9) | 0 | 1 (0.4) |
| Ligament sprain | 0 | 1 (0.9) | 1 (0.4) |
| Tibia fracture | 0 | 1 (0.9) | 1 (0.4) |
| Tooth fracture | 0 | 1 (0.9) | 1 (0.4) |
| Vascular disorders | 1 (0.9) | 3 (2.6) | 4 (1.7) |
| Hypertension | 1 (0.9) | 3 (2.6) | 4 (1.7) |
| General disorders and | 1 (0.9) | 2 (1.7) | 3 (1.3) |
| administration site conditions | | | |
| Influenza like illness | 0 | 1 (0.9) | 1 (0.4) |
| Injection site erythema | 1 (0.9) | 0 | 1 (0.4) |
| Non-cardiac chest pain | 0 | 1 (0.9) | 1 (0.4) |
| Immune system disorders | 1 (0.9) | 2 (1.7) | 3 (1.3) |
| Seasonal allergy | 1 (0.9) | 2 (1.7) | 3 (1.3) |
| Metabolism and nutrition | 1 (0.9) | 2 (1.7) | 3 (1.3) |
| disorders | | | |
| Hyperlipidaemia | 1 (0.9) | 1 (0.9) | 2 (0.9) |
| Hypertriglyceridaemia | 0 | 1 (0.9) | 1 (0.4) |
| Type 2 diabetes mellitus | 0 | 1 (0.9) | 1 (0.4) |
| Neoplasms benign, malignant and | 0 | 2 (1.7) | 2 (0.9) |
| unspecified (incl cysts and | | | |
| polyps) | | | |
| Basal cell carcinoma | 0 | 2 (1.7) | 2 (0.9) |
| Keratoacanthoma | 0 | 1 (0.9) | 1 (0.4) |
| Respiratory, thoracic and | 2 (1.8) | 0 | 2 (0.9) |
| mediastinal disorders | | | |
| Cough | 2 (1.8) | 0 | 2 (0.9) |
| Congenital, familial and genetic | 0 | 1 (0.9) | 1 (0.4) |
| disorders | | | |
| Dermoid cyst | 0 | 1 (0.9) | 1 (0.4) |
| Endocrine disorders | 0 | 1 (0.9) | 1 (0.4) |
| Hypothyroidism | 0 | 1 (0.9) | 1 (0.4) |
| Hepatobiliary disorders | 0 | 1 (0.9) | 1 (0.4) |
| Liver injury | 0 | 1 (0.9) | 1 (0.4) |
| Psychiatric disorders | 1 (0.9) | 0 | 1 (0.4 |
| Psychotic disorder | 1 (0.9) | 0 | 1 (0.4) |
| Renal and urinary disorders | 1 (0.9) | 0 | 1 (0.4) |
| Acute kidney injury | 1 (0.9) | 0 | 1 (0.4) |
| Reproductive system and breast | 0 | 1 (0.9) | 1 (0.4) |
| disorders | | | |
| Benign prostatic hyperplasia | 0 | 1 (0.9) | 1 (0.4) |

Note:
If a subject experienced more than 1 TEAE within a system organ class, the subject is counted once under that SOC. If a subject has more than 1 count of a particular preferred term, the subject was counted once for that preferred term. Part 1 (Day 1 to Week 16) includes all AEs that first occurred or worsened in severity after the first dose of study medication, but did not start after the Week 16 study medication administration.
[1] MedDRA dictionary version 24.0 was used for coding.
[2] A Treatment-emergent adverse event (TEAE) is defined as an AE that first occurred or worsened in severity after the first administration of study treatment.

TABLE 8
Subjects with Treatment-Emergent Adverse Events by System Organ Class, Preferred
Term, and Relationship to Study Drug, Part 1 Safety Analysis Set

| System Organ Class/ | Relation to | Placebo | Tildrakizumab 100 mg | All Subjects |
|---|---|---|---|---|
| Preferred Term [1] | Study Drug [2] | (N = 114) | (N = 117) | (N = 231) |
| Subjects with any | Related | 7 (6.1) | 4 (3.4) | 11 (4.8) |
| TEAE [3] | | | | |
| | Not Related | 17 (14.9) | 37 (31.6) | 54 (23.4) |
| Infections and | Related | 4 (3.5) | 1 (0.9) | 5 (2.2) |
| infestations | | | | |
| | Not Related | 2 (1.8) | 13 (11.1) | 15 (6.5) |
| Nasopharyngitis | Related | 0 | 0 | 0 |
| | Not Related | 1 (0.9) | 3 (2.6) | 4 (1.7) |
| Viral upper | Related | 0 | 1 (0.9) | 1 (0.4) |
| respiratory | | | | |
| tract infection | | | | |
| | Not Related | 1 (0.9) | 2 (1.7) | 3 (1.3) |
| Bronchitis | Related | 0 | 0 | 0 |
| | Not Related | 0 | 2 (1.7) | 2 (0.9) |
| Sinusitis | Related | 0 | 0 | 0 |
| | Not Related | 0 | 2 (1.7) | 2 (0.9) |
| Upper respiratory | Related | 1 (0.9) | 0 | 1 (0.4) |
| tract infection | | | | |
| | Not Related | 0 | 1 (0.9) | 1 (0.4) |
| Breast abscess | Related | 0 | 0 | 0 |
| | Not Related | 0 | 1 (0.9) | 1 (0.4) |
| COVID-19 | Related | 0 | 0 | 0 |
| | Not Related | 0 | 1 (0.9) | 1 (0.4) |
| Cellulitis | Related | 0 | 0 | 0 |
| | Not Related | 0 | 1 (0.9) | 1 (0.4) |
| Hordeolum | Related | 0 | 0 | 0 |
| | Not Related | 0 | 1 (0.9) | 1 (0.4) |
| Influenza | Related | 1 (0.9) | 0 | 1 (0.4) |
| | Not Related | 0 | 0 | 0 |
| Skin infection | Related | 0 | 0 | 0 |
| | Not Related | 0 | 1 (0.9) | 1 (0.4) |
| Tooth infection | Related | 0 | 0 | 0 |
| | Not Related | 1 (0.9) | 0 | 1 (0.4) |
| Urinary tract | Related | 1 (0.9) | 0 | 1 (0.4) |
| infection | | | | |
| | Not Related | 0 | 0 | 0 |
| Vulvovaginal | Related | 1 (0.9) | 0 | 1 (0.4) |
| candidiasis | | | | |
| | Not Related | 0 | 0 | 0 |
| Skin and | Related | 1 (0.9) | 2 (1.7) | 3 (1.3) |
| subcutaneous tissue | | | | |
| disorders | | | | |
| | Not Related | 1 (0.9) | 8 (6.8) | 9 (3.9) |
| Pruritus | Related | 1 (0.9) | 0 | 1 (0.4) |
| | Not Related | 1 (0.9) | 1 (0.9) | 2 (0.9) |
| Seborrhoeic | Related | 0 | 0 | 0 |
| dermatitis | | | | |
| | Not Related | 0 | 2 (1.7) | 2 (0.9) |
| Acne | Related | 0 | 0 | 0 |
| | Not Related | 0 | 1 (0.9) | 1 (0.4) |
| Guttate psoriasis | Related | 0 | 0 | 0 |
| | Not Related | 0 | 1 (0.9) | 1 (0.4) |
| Hypertrichosis | Related | 0 | 1 (0.9) | 1 (0.4) |
| | Not Related | 0 | 0 | 0 |
| Post inflammatory | Related | 0 | 0 | 0 |
| pigmentation | | | | |
| change | | | | |
| | Not Related | 0 | 1 (0.9) | 1 (0.4) |
| Rash | Related | 0 | 0 | 0 |
| | Not Related | 0 | 1 (0.9) | 1 (0.4) |
| Rash pruritic | Related | 0 | 1 (0.9) | 1 (0.4) |
| | Not Related | 0 | 0 | 0 |
| Urticaria | Related | 0 | 0 | 0 |
| | Not Related | 0 | 1 (0.9) | 1 (0.4) |
| Gastrointestinal | Related | 2 (1.8) | 0 | 2 (0.9) |
| disorders | | | | |
| | Not Related | 3 (2.6) | 4 (3.4) | 7 (3.0) |
| Diarrhoea | Related | 1 (0.9) | 0 | 1 (0.4) |
| | Not Related | 2 (1.8) | 2 (1.7) | 4 (1.7) |
| Constipation | Related | 1 (0.9) | 0 | 1 (0.4) |
| | Not Related | 0 | 0 | 0 |
| Gastrooesophageal | Related | 0 | 0 | 0 |
| reflux disease | | | | |
| | Not Related | 0 | 1 (0.9) | 1 (0.4) |
| Intra-abdominal | Related | 0 | 0 | 0 |
| haematoma | | | | |
| | Not Related | 1 (0.9) | 0 | 1 (0.4) |
| Nausea | Related | 0 | 0 | 0 |
| | Not Related | 0 | 1 (0.9) | 1 (0.4) |
| Vomiting | Related | 1 (0.9) | 0 | 1 (0.4) |
| | Not Related | 0 | 0 | 0 |
| Investigations | Related | 1 (0.9) | 1 (0.9) | 2 (0.9) |
| | Not Related | 3 (2.6) | 3 (2.6) | 6 (2.6) |
| Alanine | Related | 1 (0.9) | 0 | 1 (0.4) |
| aminotransferase | | | | |
| increased | | | | |
| | Not Related | 0 | 1 (0.9) | 1 (0.4) |
| Blood | Related | 0 | 0 | 0 |
| triglycerides | | | | |
| increased | | | | |
| | Not Related | 2 (1.8) | 0 | 2 (0.9) |
| Blood creatine | Related | 0 | 1 (0.9) | 1 (0.4) |
| phosphokinase | | | | |
| increased | | | | |
| | Not Related | 0 | 0 | 0 |
| Blood glucose | Related | 0 | 0 | 0 |
| increased | | | | |
| | Not Related | 0 | 1 (0.9) | 1 (0.4) |
| Blood potassium | Related | 0 | 0 | 0 |
| increased | | | | |
| | Not Related | 1 (0.9) | 0 | 1 (0.4) |
| SARS-CoV-2 test | Related | 0 | 0 | 0 |
| positive | | | | |
| | Not Related | 0 | 1 (0.9) | 1 (0.4) |
| Nervous system | Related | 3 (2.6) | 0 | 3 (1.3) |
| disorders | | | | |
| | Not Related | 1 (0.9) | 3 (2.6) | 4 (1.7) |
| Headache | Related | 3 (2.6) | 0 | 3 (1.3) |
| | Not Related | 1 (0.9) | 1 (0.9) | 2 (0.9) |
| Migraine | Related | 0 | 0 | 0 |
| | Not Related | 0 | 1 (0.9) | 1 (0.4) |
| Sciatica | Related | 0 | 0 | 0 |
| | Not Related | 0 | 1 (0.9) | 1 (0.4) |
| Musculoskeletal and | Related | 0 | 0 | 0 |
| connective tissue | | | | |
| disorders | | | | |
| | Not Related | 3 (2.6) | 2 (1.7) | 5 (2.2) |
| Arthralgia | Related | 0 | 0 | 0 |
| | Not Related | 1 (0.9) | 0 | 1 (0.4) |
| Intervertebral | Related | 0 | 0 | 0 |
| disc protrusion | | | | |
| | Not Related | 1 (0.9) | 0 | 1 (0.4) |
| Neck pain | Related | 0 | 0 | 0 |
| | Not Related | 0 | 1 (0.9) | 1 (0.4) |
| Pain in extremity | Related | 0 | 0 | 0 |
| | Not Related | 0 | 1 (0.9) | 1 (0.4) |
| Psoriatic | Related | 0 | 0 | 0 |
| arthropathy | | | | |
| | Not Related | 0 | 1 (0.9) | 1 (0.4) |
| Tendonitis | Related | 0 | 0 | 0 |
| | Not Related | 1 (0.9) | 0 | 1 (0.4) |
| Injury, poisoning | Related | 0 | 0 | 0 |
| and procedural | | | | |
| complications | | | | |
| | Not Related | 1 (0.9) | 3 (2.6) | 4 (1.7) |
| Foot fracture | Related | 0 | 0 | 0 |
| | Not Related | 1 (0.9) | 0 | 1 (0.4) |
| Ligament sprain | Related | 0 | 0 | 0 |
| | Not Related | 0 | 1 (0.9) | 1 (0.4) |
| Tibia fracture | Related | 0 | 0 | 0 |
| | Not Related | 0 | 1 (0.9) | 1 (0.4) |
| Tooth fracture | Related | 0 | 0 | 0 |
| | Not Related | 0 | 1 (0.9) | 1 (0.4) |
| Vascular disorders | Related | 0 | 0 | 0 |
| | Not Related | 1 (0.9) | 3 (2.6) | 4 (1.7) |
| Hypertension | Related | 0 | 0 | 0 |
| | Not Related | 1 (0.9) | 3 (2.6) | 4 (1.7) |
| General disorders | Related | 1 (0.9) | 0 | 1 (0.4) |
| and administration | | | | |
| site conditions | | | | |
| | Not Related | 0 | 2 (1.7) | 2 (0.9) |
| Influenza like | Related | 0 | 0 | 0 |
| illness | | | | |
| | Not Related | 0 | 1 (0.9) | 1 (0.4) |
| Injection site | Related | 1 (0.9) | 0 | 1 (0.4) |
| erythema | | | | |
| | Not Related | 0 | 0 | 0 |
| Non-cardiac chest | Related | 0 | 0 | 0 |
| pain | | | | |
| | Not Related | 0 | 1 (0.9) | 1 (0.4) |
| Immune system | Related | 0 | 0 | 0 |
| disorders | | | | |
| | Not Related | 1 (0.9) | 2 (1.7) | 3 (1.3) |
| Seasonal allergy | Related | 0 | 0 | 0 |
| | Not Related | 1 (0.9) | 2 (1.7) | 3 (1.3) |
| Metabolism and | Related | 0 | 0 | 0 |
| nutrition disorders | | | | |
| | Not Related | 1 (0.9) | 2 (1.7) | 3 (1.3) |
| Hyperlipidaemia | Related | 0 | 0 | 0 |
| | Not Related | 1 (0.9) | 1 (0.9) | 2 (0.9) |
| Hypertriglyceridaemia | Related | 0 | 0 | 0 |
| | Not Related | 0 | 1 (0.9) | 1 (0.4) |
| Type 2 diabetes | Related | 0 | 0 | 0 |
| mellitus | | | | |
| | Not Related | 0 | 1 (0.9) | 1 (0.4) |
| Neoplasms benign, | Related | 0 | 0 | 0 |
| malignant and | | | | |
| unspecified (incl | | | | |
| cysts and polyps) | | | | |
| | Not Related | 0 | 2 (1.7) | 2 (0.9) |
| Basal cell | Related | 0 | 0 | 0 |
| carcinoma | | | | |
| | Not Related | 0 | 2 (1.7) | 2 (0.9) |
| Keratoacanthoma | Related | 0 | 0 | 0 |
| | Not Related | 0 | 1 (0.9) | 1 (0.4) |
| Respiratory, | Related | 0 | 0 | 0 |
| thoracic and | | | | |
| mediastinal | | | | |
| disorders | | | | |
| | Not Related | 2 (1.8) | 0 | 2 (0.9) |
| Cough | Related | 0 | 0 | 0 |
| | Not Related | 2 (1.8) | 0 | 2 (0.9) |
| Congenital, familial | Related | 0 | 0 | 0 |
| and genetic | | | | |
| disorders | | | | |
| | Not Related | 0 | 1 (0.9) | 1 (0.4) |
| Dermoid cyst | Related | 0 | 0 | 0 |
| | Not Related | 0 | 1 (0.9) | 1 (0.4) |
| Endocrine disorders | Related | 0 | 0 | 0 |
| | Not Related | 0 | 1 (0.9) | 1 (0.4) |
| Hypothyroidism | Related | 0 | 0 | 0 |
| | Not Related | 0 | 1 (0.9) | 1 (0.4) |
| Hepatobiliary | Related | 0 | 0 | 0 |
| disorders | | | | |
| | Not Related | 0 | 1 (0.9) | 1 (0.4) |
| Liver injury | Related | 0 | 0 | 0 |
| | Not Related | 0 | 1 (0.9) | 1 (0.4) |
| Psychiatric | Related | 0 | 0 | 0 |
| disorders | | | | |
| | Not Related | 1 (0.9) | 0 | 1 (0.4) |
| Psychotic | Related | 0 | 0 | 0 |
| disorder | | | | |
| | Not Related | 1 (0.9) | 0 | 1 (0.4) |
| Renal and urinary | Related | 0 | 0 | 0 |
| disorders | | | | |
| | Not Related | 1 (0.9) | 0 | 1 (0.4) |
| Acute kidney | Related | 0 | 0 | 0 |
| injury | | | | |
| | Not Related | 1 (0.9) | 0 | 1 (0.4) |
| Reproductive system | Related | 0 | 0 | 0 |
| and breast disorders | | | | |
| | Not Related | 0 | 1 (0.9) | 1 (0.4) |
| Benign prostatic | Related | 0 | 0 | 0 |
| hyperplasia | | | | |
| | Not Related | 0 | 1 (0.9) | 1 (0.4) |

Note:
Part 1 (Day 1 to Week 16) includes all AEs that first occurred or worsened in severity after the first dose of study medication, but did not start after the Week 16 study medication administration. If a subject experienced more than 1 relationship within a SOC or PT, the subject is counted once under maximum causality. If an AE is missing the relationship to study medication, the event will be assumed to be related for analysis and summarization.
[1] MedDRA dictionary version 24.0 was used for coding
[2] Related = includes probably, possibly, and certainly/definitely study drug related; Not Related = includes unlikely and unrelated.
[3] A Treatment-emergent adverse event (TEAE) is defined as an AE that first occurred or worsened in severity after the first administration of study treatment.

TABLE 9
Extent of Exposure, Part 1 SAF

| Dose Administered | | | |
|---|---|---|---|
| at Each Visit, n(%) | Placebo | Tildrakizumab 100 mg | All Subjects |
| Part 1 | (N = 114) | (N = 117) | (N = 231) |
| Baseline/Week 0 | 114 (100.0) | 117 (100.0) | 231 (100.0) |
| Week 4 | 111 (97.4) | 115 (98.3) | 226 (97.8) |
| Subjects Received | 111 (97.4) | 115 (98.3) | 226 (97.8) |
| Both Planned Doses | | | |
| in Part 1, n(%) | | | |

Note:
Treatment group is the treatment received in Part 1.

TABLE 10
Analysis of Primary Efficacy Outcome - IGA Mod
2011 (Scalp) Response Rate at Week 16 mITT, NRI

| | Placebo | Tildrakizumab 100 mg |
|---|---|---|
| | (N = 82) | (N = 89) |
| IGA Mod 2011(Scalp) Response | | |
| Rate at Week 16 | | |
| n/N (%) | 6/82 (7.3) | 44/89 (49.4) |
| 95% CI | 2.73, 15.25 | 38.67, 60.25 |
| Statistical Test [1] | | |
| Response Rate Difference, | | 42.2 |
| Tildrakizumab - Placebo (%) | | |
| 95% CI | | 30.42, 54.06 |
| p-value [2] | | <0.0001 |
| Proportion of PSSI-90 | | |
| Week 16 Responders with | | |
| Maintained Response | | |
| through Week 52/EOT | | |
| N′ | 6 | 44 |
| n/N (%) | 4/6 (66.7) | 36/44 (81.8) |
| 95% CI | 22.28, 95.67 | 67.29, 91.81 |
| Statistical Test [1] | | |
| Response Rate Difference, | | 12.9 |
| Tildrakizumab - Placebo (%) | | |
| 95% CI | | −27.56, 53.29 |
| p-value [2] | | 0.49556 |

Note:
IGA = Investigator Global Assessment. IGA Mod 2011 (scalp) response is defined as an IGA score of “clear” in subjects enrolled under Protocol Amendment 01 and “clear” and “almost clear” in subjects enrolled under Protocol Amendment 02, or 03, in both cases with at least 2-point reduction from baseline. mITT = Modified ITT Set. NRI = Non-responder imputation. CI = confidence interval.
[1] Response Rate Difference and CI are calculated using Miettinen-Nurminen, stratified by body weight (<=90 kg, >90 kg) and prior exposure to TNF-alpha inhibitors (yes/no).
[2] P-value is calculated using the Cochran-Mantel-Haenszel (CMH) test stratified by body weight (<=90 kg, >90 kg) and prior exposure to TNF-alpha inhibitors (yes/no). P-value is not adjusted for multiplicity.

TABLE 11
Analysis of Primary Efficacy Outcome - IGA Mod
2011 (Scalp) Response Rate at Week 16 uITT, NRI

| | Placebo | Tildrakizumab 100 mg |
|---|---|---|
| | (N = 45) | (N = 44) |
| IGA Mod 2011 (Scalp) Response | | |
| Rate at Week 16 | | |
| n/N (%) | 5/45 (11.1) | 23/44 (52.3) |
| 95% CI | 3.71, 24.05 | 36.69, 67.54 |
| Statistical Test [1] | | |
| Response Rate Difference, | | 35.5 |
| Tildrakizumab - Placebo (%) | | |
| 95% CI | | 18.91, 52.07 |
| p-value [2] | | <0.001 |

Note:
IGA = Investigator Global Assessment. IGA Mod 2011 (scalp) response is defined as an IGA score of “clear” in subjects enrolled under Protocol Amendment 01 and “clear” and “almost clear” in subjects enrolled under Protocol Amendment 02, or 03, in both cases with at least 2-point reduction from baseline. uITT = Uniform Intent to Treat Set. NRI = Non-responder imputation. CI = confidence interval.
[1] Response Rate Difference and CI are calculated using Miettinen-Nurminen, stratified by body weight (<=90 kg, >90 kg) and prior exposure to TNF-alpha inhibitors (yes/no).
[2] P-value is calculated using the Cochran-Mantel-Haenszel (CMH) test stratified by body weight (<=90 kg, >90 kg) and prior exposure to TNF-alpha inhibitors (yes/no). P-value is not adjusted for multiplicity.

TABLE 12
Analysis of Primary Efficacy Outcome - IGA Mod
2011 (Scalp) Response Rate at Week 16 PP, OC

| | Placebo | Tildrakizumab 100 mg |
|---|---|---|
| | (N = 75) | (N = 80) |
| IGA Mod 2011(Scalp) Response | | |
| Rate at Week 16 | | |
| n/N (%) | 5/75 (6.7) | 43/80 (53.8) |
| 95% CI | 2.20, 14.88 | 42.24, 64.97 |
| Statistical Test [1] | | |
| Response Rate Difference, | | 44.2 |
| Tildrakizumab - Placebo (%) | | |
| 95% CI | | 31.97, 56.50 |
| p-value [2] | | <0.001 |

Note:
IGA = Investigator Global Assessment. IGA Mod 2011 (scalp) response is defined as an IGA score of “clear” in subjects enrolled under Protocol Amendment 01 and “clear” and “almost clear” in subjects enrolled under Protocol Amendment 02, or 03, in both cases with at least 2-point reduction from baseline. PP = Per Protocol Set. OC = Observed Cases. CI = confidence interval.
[1] Response Rate Difference and CI are calculated using Miettinen-Nurminen, stratified by body weight (<=90 kg, >90 kg) and prior exposure to TNF-alpha inhibitors (yes/no).
[2] P-value is calculated using the Cochran-Mantel-Haenszel (CMH) test stratified by body weight (<=90 kg, >90 kg) and prior exposure to TNF-alpha inhibitors (yes/no). P-value is not adjusted for multiplicity.

TABLE 13
Analysis of Key Secondary Efficacy Outcome -
PSSI-90 Response Rate at Week 16 ITT, NRI

| | Placebo | Tildrakizumab 100 mg |
|---|---|---|
| | (N = 114) | (N = 117) |
| PSSI-90 Response | | |
| Rate at Week 16 | | |
| n/N (%) | 5/114 (4.4) | 66/117 (56.4) |
| 95% CI | 1.44, 9.94 | 46.93, 65.55 |
| Statistical Test [1] | | |
| Response Rate Difference, | | 52.1 |
| Tildrakizumab - Placebo (%) | | |
| 95% CI | | 42.42, 61.75 |
| p-value [2] | | <0.00001 |
| Proportion of PSSI-90 | | |
| Week 16 Responders with | | |
| Maintained Response | | |
| through Week 52/EOT | | |
| N′ | 5 | 66 |
| n/N (%) | 5/5 (100) | 56/66 (84.8) |
| 95% CI | 47.82, 100.00 | 73.90, 92.49 |
| Statistical Test [1] | | |
| Response Rate Difference, | | −16.6 |
| Tildrakizumab - Placebo (%) | | |
| 95% CI | | −27.85, −5.34 |
| p-value [2] | | 0.32855 |

Note:
PSSI = Psoriasis Scalp Severity Index. PSSI-90 response is defined as achieving a reduction of 90% or greater from baseline. NRI = Non-responder imputation. CI = confidence interval.
[1] Response Rate Difference and CI are calculated using Miettinen-Nurminen, stratified by body weight (<=90 kg, >90 kg) and prior exposure to TNF-alpha inhibitors (yes/no).
[2] P-value is calculated using the Cochran-Mantel-Haenszel (CMH) test stratified by body weight (<=90 kg, >90 kg) and prior exposure to TNF-alpha inhibitors (yes/no). P-value is not adjusted for multiplicity.

TABLE 14
Analysis of Key Secondary Efficacy Outcome -
PSSI-90 Response Rate at Week 16 mITT, NRI

| | Placebo | Tildrakizumab 100 mg |
|---|---|---|
| | (N = 82) | (N = 89) |
| PSSI-90 Response | | |
| Rate at Week 16 | | |
| n/N (%) | 4/82 (4.9) | 54/89 (60.7) |
| 95% CI | 1.34, 12.02 | 49.75, 70.87 |
| Statistical Test [1] | | |
| Response Rate Difference, | | 53.0 |
| Tildrakizumab - Placebo (%) | | |
| 95% CI | | 42.32, 63.66 |
| p-value [2] | | <0.001 |

Note:
PSSI = Psoriasis Scalp Severity Index. PSSI-90 response is defined as achieving a reduction of 90% or greater from baseline. mITT = Modified ITT. NRI = Non-responder imputation. CI = confidence interval.
[1] Response Rate Difference and CI are calculated using Miettinen-Nurminen, stratified by body weight (<=90 kg, >90 kg) and prior exposure to TNF-alpha inhibitors (yes/no).
[2] P-value is calculated using the Cochran-Mantel-Haenszel (CMH) test stratified by body weight (<=90 kg, >90 kg) and prior exposure to TNF-alpha inhibitors (yes/no). P-value is not adjusted for multiplicity.

TABLE 15
Analysis of Secondary Efficacy Outcome - IGA
(Scalp) Response Rate at Week 16 ITT, NRI

| | Placebo | Tildrakizumab 100 mg |
|---|---|---|
| | (N = 114) | (N = 117) |
| IGA (Scalp) Response | | |
| Rate at Week 16 | | |
| n/N (%) | 12/113 (10.6) | 80/117 (68.4) |
| 95% CI | 5.61, 17.82 | 59.13, 76.66 |
| Statistical Test [1] | | |
| Response Rate Difference, | | 57.7 |
| Tildrakizumab | | |
| Placebo (%) | | |
| 95% CI | | 47.56, 67.89 |
| p-value | | <0.001 |

Note:
IGA = Investigator Global Assessment. IGA (Scalp) Response is defined as achieving IGA (scalp only) score of “clear” and “almost clear” with at least 2-point reduction from Baseline at Week 16. NRI = Non-responder imputation. CI = confidence interval.
[1] Response Rate Difference, CI, and p-value are calculated using the Cochran-Mantel-Haenszel (CMH) test stratified by body weight class (<=90 kg, >90 kg) and prior exposure to TNF-alpha inhibitors (yes/no). P-value is not adjusted for multiplicity.

TABLE 16
Analysis of Secondary Efficacy Outcome - IGA
(Scalp) Response Rate at Week 16 mITT, NRI

| | Placebo | Tildrakizumab 100 mg |
|---|---|---|
| | (N = 82) | (N = 89) |
| IGA (Scalp) Response | | |
| Rate at Week 16 | | |
| n/N (%) | 10/81 (12.3) | 63/89 (70.8) |
| 95% CI | 6.08, 21.53 | 60.19, 79.95 |
| Statistical Test [1] | | |
| Response Rate Difference, | | 58.5 |
| Tildrakizumab | | |
| Placebo (%) | | |
| 95% CI | | 46.57, 70.35 |
| p-value | | <0.001 |

Note:
IGA = Investigator Global Assessment. IGA (Scalp) Response is defined as achieving IGA (scalp only) score of “clear” and “almost clear” with at least 2-point reduction from Baseline at Week 16. mITT = Modified Intent to Treat Set. NRI = Non-responder imputation. CI = confidence interval.
[1] Response Rate Difference, CI, and p-value are calculated using the Cochran-Mantel-Haenszel (CMH) test stratified by body weight class (<=90 kg, >90 kg) and prior exposure to TNF-alpha inhibitors (yes/no). P-value is not adjusted for multiplicity.

TABLE 17
Analysis of Key Secondary Efficacy Outcome - IGA Mod
2011 (Scalp) Response Rate at Week 12 mITT, NRI

| | Placebo | Tildrakizumab 100 mg |
|---|---|---|
| | (N = 82) | (N = 89) |
| IGA Mod 2011(Scalp) Response | | |
| Rate at Week 12 | | |
| n/N (%) | 4/82 (4.9) | 41/89 (46.1) |
| 95% CI | 1.34, 12.02 | 35.44, 56.96 |
| Statistical Test [1] | | |
| Response Rate Difference, | | 41.2 |
| Tildrakizumab | | |
| Placebo (%) | | |
| 95% CI | | 29.77, 52.53 |
| p-value [2] | | <0.00001 |

Note:
IGA = Investigator Global Assessment. IGA Mod 2011 (scalp) response is defined as an IGA score of “clear” in subjects enrolled under Protocol Amendment 01 and “clear” and “almost clear” in subjects enrolled under Protocol Amendment 02, or 03, in both cases with at least 2-point reduction from baseline. mITT = Modified ITT Set. NRI = Non-responder imputation. CI = confidence interval.
[1] Response Rate Difference and CI are calculated using Miettinen-Nurminen, stratified by body weight (<=90 kg, >90 kg) and prior exposure to TNF-alpha inhibitors (yes/no).
[2] P-value is calculated using the Cochran-Mantel-Haenszel (CMH) test stratified by body weight (<=90 kg, >90 kg) and prior exposure to TNF-alpha inhibitors (yes/no). P-value is not adjusted for multiplicity.

TABLE 18
Analysis of Exploratory Efficacy Outcome - IGA Mod
2011 (Scalp) Response Rate by Visit mITT, NRI

| | Placebo | | Tildrakizumab 100 mg |
|---|---|---|---|
| e | (N = 82) | | (N = 89) |
| IGA Mod (Scalp) Response Rate at Week 1 | | | |
| n/N (%) | 0/82 | (0.0) | 1/89 (1.1) |
| 95% CI | 0.00, 4.40 | | 0.03, 6.10 |
| Cochran-Mantel-Haenszel (CMH) Test [1] | | | |
| Response Rate Difference (%), | | | 1.1 |
| Tildrakizumab - Placebo | | | |
| 95% CI | | | −1.07, 3.30 |
| p-value | | | 0.34092 |
| IGA Mod (Scalp) Response Rate at Week 4 | | | |
| n/N (%) | 1/82 | (1.2) | 11/89 (12.4) |
| 95% CI | 0.03, 6.61 | | 6.33, 21.04 |
| Cochran-Mantel-Haenszel (CMH) Test [1] | | | |
| Response Rate Difference (%), | | | 11.1 |
| Tildrakizumab - Placebo | | | |
| 95% CI | | | 3.80, 18.32 |
| p-value | | | 0.00439 |
| IGA Mod (Scalp) Response Rate at Week 8 | | | |
| n/N (%) | 2/82 | (2.4) | 29/89 (32.6) |
| 95% CI | 0.30, 8.53 | | 23.02, 43.34 |
| Cochran-Mantel-Haenszel (CMH) Test [1] | | | |
| Response Rate Difference (%), | | | 30.3 |
| Tildrakizumab - Placebo | | | |
| 95% CI | | | 20.03, 40.63 |
| p-value | | | <0.00001 |
| IGA Mod (Scalp) Response Rate at Week 12 | | | |
| n/N (%) | 4/82 | (4.9) | 41/89 (46.1) |
| 95% CI | 1.34, 12.02 | | 35.44, 56.96 |
| Cochran-Mantel-Haenszel (CMH) Test [1] | | | |
| Response Rate Difference (%), | | | 41.2 |
| Tildrakizumab - Placebo | | | |
| 95% CI | | | 29.77, 52.53 |
| p-value | | | <0.00001 |
| IGA Mod (Scalp) Response Rate at Week 16 | | | |
| n/N (%) | 6/82 | (7.3) | 44/89 (49.4) |
| 95% CI | 2.73, 15.25 | | 38.67, 60.25 |
| Cochran-Mantel-Haenszel (CMH) Test [1] | | | |
| Response Rate Difference (%), | | | 42.2 |
| Tildrakizumab - Placebo | | | |
| 95% CI | | | 30.42, 54.06 |
| p-value | | | <0.00001 |
| IGA Mod (Scalp) Response Rate at Week 20 | | | |
| n/N (%) | 23/82 | (28.0) | 54/89 (60.7) |
| 95% CI | 18.68, 39.06 | | 49.75, 70.87 |
| IGA Mod (Scalp) Response Rate at Week 24 | | | |
| n/N (%) | 38/82 | (46.3) | 53/89 (59.6) |
| 95% CI | 35.25, 57.70 | | 48.62, 69.83 |
| IGA Mod (Scalp) Response Rate at Week 28 | | | |
| n/N (%) | 38/82 | (46.3) | 53/89 (59.6) |
| 95% CI | 35.25, 57.70 | | 48.62, 69.83 |
| IGA Mod (Scalp) Response Rate at Week 32 | | | |
| n/N (%) | 44/82 | (53.7) | 57/89 (64.0) |
| 95% CI | 42.30, 64.75 | | 53.18, 73.95 |
| IGA Mod (Scalp) Response Rate at Week 40 | | | |
| n/N (%) | 43/82 | (52.4) | 57/89 (64.0) |
| 95% CI | 41.11, 63.59 | | 53.18, 73.95 |
| IGA Mod (Scalp) Response Rate at Week 44 | | | |
| n/N (%) | 46/82 | (56.1) | 55/89 (61.8) |
| 95% CI | 44.70, 67.04 | | 50.89, 71.90 |
| IGA Mod (Scalp) Response Rate at Week 52/EOT | | | |
| n/N (%) | 46/82 | (56.1) | 56/89 (62.9) |

| | | |
|---|---|---|
| 95% CI | 44.70, 67.04 | 52.03, 72.93 |

Note:
IGA = Investigator Global Assessment. IGA Mod 2011 (scalp) response is defined as an IGA score of “clear” in subjects enrolled in Protocol Amendment 01, and as an IGA score of “clear” or “almost clear” in subjects enrolled in Protocol Amendments 02 or 03, in both cases with at least 2-point reduction from baseline.
mITT = Modified ITT Set. NRI = Non-responder imputation. CI = confidence interval. mITT = Modified ITT Set. NRI = Non-responder imputation. CI = confidence interval.
[1] CMH test is stratified by body weight class (<=90 kg or >90 kg) and prior exposure to TNF-alpha inhibitors (Yes/No).

TABLE 19
Analysis of Exploratory Efficacy Outcome -
PSSI 90 Response Rate by Visit ITT, NRI

| | Placebo | Tildrakizumab 100 mg |
|---|---|---|
| | (N = 114) | (N = 117) |
| PSSI-90 Response Rate at Week 1 | | |
| n/N (%) | 0/114 (0.0) | 2/117 (1.7) |
| 95% CI | 0.00, 3.18 | 0.21, 6.04 |
| Cochran-Mantel-Haenszel (CMH) Test [1] | | |
| Response Rate Difference (%), | | 1.7 |
| Tildrakizumab - Placebo | | |
| 95% CI | | −0.65, 4.01 |
| p-value | | 0.16950 |
| PSSI-90 Response Rate at Week 4 | | |
| n/N (%) | 0/114 (0.0) | 13/117 (11.1) |
| 95% CI | 0.00, 3.18 | 6.05, 18.25 |
| Cochran-Mantel-Haenszel (CMH) Test [1] | | |
| Response Rate Difference (%), | | 11.0 |
| Tildrakizumab - Placebo | | |
| 95% CI | | 5.31, 16.65 |
| p-value | | 0.00029 |
| PSSI-90 Response Rate at Week 8 | | |
| n/N (%) | 3/114 (2.6) | 43/117 (36.8) |
| 95% CI | 0.55, 7.50 | 28.03, 46.16 |
| Cochran-Mantel-Haenszel (CMH) Test [1] | | |
| Response Rate Difference (%), | | 33.9 |
| Tildrakizumab - Placebo | | |
| 95% CI | | 24.69, 43.13 |
| p-value | | <0.00001 |
| PSSI-90 Response Rate at Week 12 | | |
| n/N (%) | 2/114 (1.8) | 50/117 (42.7) |
| 95% CI | 0.21, 6.19 | 33.63, 52.21 |
| Cochran-Mantel-Haenszel (CMH) Test [1] | | |
| Response Rate Difference (%), | | 40.8 |
| Tildrakizumab - Placebo | | |
| 95% CI | | 31.57, 50.13 |
| p-value | | <0.00001 |
| PSSI-90 Response Rate at Week 16 | | |
| n/N (%) | 5/114 (4.4) | 66/117 (56.4) |
| 95% CI | 1.44, 9.94 | 46.93, 65.55 |
| Cochran-Mantel-Haenszel (CMH) Test [1] | | |
| Response Rate Difference (%), | | 52.1 |
| Tildrakizumab - Placebo | | |
| 95% CI | | 42.42, 61.75 |
| p-value | | <0.00001 |
| PSSI-90 Response Rate at Week 20 | | |
| n/N (%) | 16/114 (14.0) | 72/117 (61.5) |
| 95% CI | 8.24, 21.79 | 52.09, 70.38 |
| PSSI-90 Response Rate at Week 24 | | |
| n/N (%) | 43/114 (37.7) | 73/117 (62.4) |
| 95% CI | 28.81, 47.28 | 52.96, 71.18 |
| PSSI-90 Response Rate at Week 28 | | |
| n/N (%) | 58/114 (50.9) | 73/117 (62.4) |
| 95% CI | 41.35, 60.36 | 52.96, 71.18 |
| PSSI-90 Response Rate at Week 32 | | |
| n/N (%) | 67/114 (58.8) | 75/117 (64.1) |
| 95% CI | 49.17, 67.91 | 54.71, 72.76 |
| PSSI-90 Response Rate at Week 40 | | |
| n/N (%) | 70/114 (61.4) | 77/117 (65.8) |
| 95% CI | 51.83, 70.37 | 56.47, 74.33 |
| PSSI-90 Response Rate at Week 44 | | |
| n/N (%) | 70/114 (61.4) | 72/117 (61.5) |
| 95% CI | 51.83, 70.37 | 52.09, 70.38 |
| PSSI-90 Response Rate at Week 52/EOT | | |
| n/N (%) | 68/114 (59.6) | 77/117 (65.8) |
| 95% CI | 50.05, 68.73 | 56.47, 74.33 |

Note:
PSSI = Psoriasis Scalp Severity Index. PSSI-90 response is defined as achieving a reduction of 90% or greater from baseline. NRI = Non-responder imputation. CI = confidence interval.
[1] CMH test is stratified by body weight class (=90 kg or >90 kg) and prior exposure to TNF-alpha inhibitors (Yes/No).

TABLE 20
Proportion of Subjects with Investigator Global Assessment
Mod 2011 (Scalp) Score of “Clear” and “Almost
Clear” with at Least 2-point Reduction from Baseline at Week 16

| | Placebo | Tildrakizumab |
|---|---|---|
| Number of Participants Analyzed | 82 | 89 |
| The Proportion of Subjects with Investigator | 0.073 | 0.494 |
| Global Assessment Mod 2011 (Scalp) Score of | | p-value: |
| “Clear” and “Almost Clear” with at Least 2- | | <0.00001 |
| point Reduction from Baseline at Week 16 | | |

Subjects will receive Placebo injections up to week 4. At Week 16, all the subjects from this arm were switched to receive Tildrakizumab up to week 44.

TABLE 21
Proportion of Subjects with at Least 90% Improvement From
Baseline in the Psoriasis Scalp Severity Index at Week 16

| | Placebo | Tildrakizumab |
|---|---|---|
| Number of Participants Analyzed | 114 | 117 |
| The Proportion of Subjects with at Least 90% | 0.044 | 0.56 |
| Improvement From Baseline in the Psoriasis | | p-value: |
| Scalp Severity Index at Week 16 | | <0.00001 |

Subjects will receive Placebo injections up to week 4. At Week 16, all the subjects from this arm were switched to receive Tildrakizumab up to week 44.

TABLE 22
Mean Percentage Change in Psoriasis Scalp Severity
Index Score From Baseline to Week 16.

| | Placebo | Tildrakizumab |
|---|---|---|
| Number of Participants Analyzed | 107 | 110 |
| Mean Percentage Change in | −21.84 | −79.81 |
| Psoriasis Scalp | (35.577) | (30.434) |
| Severity Index Score From | | p-value: |
| Baseline to Week 16. | | <0.00001 |

Subjects will receive Placebo injections up to week 4. At Week 16, all the subjects from this arm were switched to receive Tildrakizumab up to week 44.

TABLE 23
Proportion of Subjects Achieving Psoriasis
Scalp Severity Index 75 at Week 16

| | Placebo | Tildrakizumab |
|---|---|---|
| Number of Participants Analyzed | 114 | 117 |
| Proportion of Subjects Achieving Psoriasis | 0.105 | 0.709 |
| Scalp Severity Index 75 at Week 16 | | p-value: |
| | | <0.00001 |

Subjects will receive Placebo injections up to week 4. At Week 16, all the subjects from this arm were switched to receive Tildrakizumab up to week 44.

TABLE 24
Proportion of Subjects Achieving Psoriasis
Scalp Severity Index 100 at Week 16

| | Placebo | Tildrakizumab |
|---|---|---|
| Number of Participants Analyzed | 114 | 117 |
| Proportion of Subjects Achieving Psoriasis | 0.026 | 0.376 |
| Scalp Severity Index 100 at Week 16 | | p-value: |
| | | <0.00001 |

Subjects will receive Placebo injections up to week 4. At Week 16, all the subjects from this arm were switched to receive Tildrakizumab up to week 44.

TABLE 25
Mean Percentage Change in Scalp Surface Area
(SSA) Involvement From Baseline to Week 16

| | Placebo | Tildrakizumab |
|---|---|---|
| Number of Participants Analyzed | 107 | 110 |
| Mean Percentage Change in Scalp | −15.2 | −76.39 |
| Surface Area (SSA) | (31.473) | (35.179) |
| Involvement From Baseline | | p-value: |
| to Week 16 | | <0.00001 |

Subjects will receive Placebo injections up to week 4. At Week 16, all the subjects from this arm were switched to receive Tildrakizumab up to week 44.

TABLE 26
Time to 75% Reduction in Psoriasis Scalp Severity Index
During 16-week Placebo-controlled Treatment Period.

| | Placebo | Tildrakizumab |
|---|---|---|
| Number of Participants Analyzed | 114 | 117 |
| Time to 75% Reduction in Psoriasis Scalp | 17 | 86 |
| Severity Index During 16-week Placebo- | (14.91%) | (73.5%) |
| controlled Treatment Period. | | p-value: |
| | | <0.00001 |

Subjects will receive Placebo injections up to week 4. At Week 16, all the subjects from this arm were switched to receive Tildrakizumab up to week 44.

TABLE 27
Time to Investigator Global Assessment Mod 2011 (Scalp) Response
During the 16-week Placebo-controlled Treatment Period.

| | Placebo | Tildrakizumab |
|---|---|---|
| Number of Participants Analyzed | 82 | 89 |
| Time to Investigator Global | 6 | 55 |
| Assessment Mod 2011 (Scalp) | (7.32%) | (61.8%) |
| Response During the 16-week | | p-value: |
| Placebo controlled Treatment Period. | | <0.00001 |

Subjects will receive Placebo injections up to week 4. At Week 16, all the subjects from this arm were switched to receive Tildrakizumab up to week 44.

TABLE 28
Proportion of Subjects Achieving a 4-point Reduction in
Itch Numeric Rating Scale Score from Baseline to Week 16

| | Placebo | Tildrakizumab |
|---|---|---|
| Number of Participants Analyzed | 102 | 101 |
| Proportion of Subjects Achieving a 4-point | 0.147 | 0.545 |
| Reduction in Itch Numeric Rating Scale Score | | p-value: |
| from Baseline to Week 16 | | <0.00001 |

Subjects will receive Placebo injections up to week 4. At Week 16, all the subjects from this arm were switched to receive Tildrakizumab up to week 44.

TABLE 29
Proportion of Subjects Achieving Psoriasis Area and Severity
Index (PASI) 75, Psoriasis Area and Severity Index 90,
and Psoriasis Area and Severity Index 100 at Week 16

| | Placebo | Tildrakizumab | |
|---|---|---|---|
| Number of Participants Analyzed | 114 | 117 | |
| PASI-75 | 0.079 | 0.667 | (p-value <0.00001) |
| PASI-90 | 0.026 | 0.41 | (p-value <0.00001) |
| PASI-100 | 0.009 | 0.162 | (p-value 0.0004) |

Subjects will receive Placebo injections up to week 4. At Week 16, all the subjects from this arm were switched to receive Tildrakizumab up to week 44.

TABLE 30
Proportion of Subjects with Physician's Global Assessment
Score (Whole Body) Score of “Clear” or “Almost
Clear” with at Least a 2-point Reduction from Baseline to Week 16

| | Placebo | Tildrakizumab |
|---|---|---|
| Number of Participants Analyzed | 114 | 117 |
| Proportion of Subjects with Physician's Global | 0.062 | 0.556 |
| Assessment Score (Whole Body) Score | | p-value: |
| of “Clear” or “Almost Clear” | | <0.00001 |
| with at Least a 2-point Reduction from | | |
| Baseline to Week 16 | | |

Subjects will receive Placebo injections up to week 4. At Week 16, all the subjects from this arm were switched to receive Tildrakizumab up to week 44.

TABLE 31
Mean Percentage Change in Total Body Surface Area
(BSA) Involvement from Baseline to Week 16

| | Placebo | Tildrakizumab |
|---|---|---|
| Number of Participants Analyzed | 107 | 110 |
| Mean Percentage Change in Total | 0.19 | −70.17 |
| Body Surface Area (BSA) Involvement | (39.039) | (33.443) |
| from Baseline to Week 16 | | p-value: |
| | | <0.00001 |

Subjects will receive Placebo injections up to week 4. At Week 16, all the subjects from this arm were switched to receive Tildrakizumab up to week 44.

TABLE 32
Proportion of Subjects with Investigator Global Assessment
(Scalp Only) Score of “Clear” and “Almost Clear”
with at Least 2-point Reduction from Baseline at Week 16

| | Placebo | Tildrakizumab |
|---|---|---|
| Number of Participants Analyzed | 114 | 117 |
| Proportion of Subjects with Investigator Global | 0.106 | 0.684 |
| Assessment (Scalp Only) Score of “Clear” and | | p-value: |
| “Almost Clear” with at Least 2-point Reduction | | <0.00001 |
| from Baseline at Week 16. | | |

Subjects will receive Placebo injections up to week 4. At Week 16, all the subjects from this arm were switched to receive Tildrakizumab up to week 44.

TABLE 33
Proportion of Subjects with Investigator Global Assessment
Mod 2011 Score (Whole Body) of “Clear” and “Almost
Clear” with at Least 2-point Reduction from Baseline at Week 16

| | Placebo | Tildrakizumab |
|---|---|---|
| Number of Participants Analyzed | 82 | 89 |
| Proportion of Subjects with Investigator Global | 0.038 | 0.545 |
| Assessment Mod 2011 Score (Whole Body) of | | p-value: |
| “Clear” and “Almost Clear” with at Least 2- | | <0.00001 |
| point Reduction from Baseline at Week 16. | | |

Subjects will receive Placebo injections up to week 4. At Week 16, all the subjects from this arm were switched to receive Tildrakizumab up to week 44.

TABLE 34
Proportion of Subjects with IGA Mod 2011 (Scalp) Score of “Clear” and
“Almost Clear” with at Least 2-point Reduction from Baseline

| | Placebo | Tildrakizumab |
|---|---|---|
| Number of Participants Analyzed | 82 | 89 |
| Proportion of Subjects with IGA Mod 2011 | 0.049 | 0.461 |
| (Scalp) Score of “Clear” and “Almost Clear” | | p-value: |
| with at Least 2-point Reduction from Baseline. | | <0.00001 |

Subjects will receive Placebo injections up to week 4. At Week 16, all the subjects from this arm were switched to receive Tildrakizumab up to week 44.

TABLE 35
Proportion of Subjects with at Least 90% Improvement from
Baseline in the Psoriasis Scalp Severity Index at Week 12

| | Placebo | Tildrakizumab |
|---|---|---|
| Number of Participants Analyzed | 114 | 117 |
| Proportion of Subjects with at Least 90% | 0.018 | 0.427 |
| Improvement from Baseline in the Psoriasis | | p-value: |
| Scalp Severity Index at Week 12. | | <0.00001 |

Subjects will receive Placebo injections up to week 4. At Week 16, all the subjects from this arm were switched to receive Tildrakizumab up to week 44.

TABLE 36
Change in Investigator Global Assessment
Mod 2011 (Scalp) from Baseline at Week 52

| | Placebo | Tildrakizumab |
|---|---|---|
| Number of Participants Analyzed | 68 | 77 |
| 1 = worsen by 1 | 0 | 0 |
| 0 = no change | 1 | 2 |
| −1 = improve by 1 | 14 | 13 |
| −2 = improve by 2 | 9 | 9 |
| −3 = improve by 3 | 38 | 48 |
| −4 = improve by 4 | 6 | 5 |

Subjects received Placebo injections up to week 4, then at Week 16, all the subjects from Placebo arm were switched to receive Tildrakizumab up to week 44.

TABLE 37
Change in IGA Mod 2011 (Whole-
body) From Baseline at Week 52

| | Placebo | | Tildrakizumab | |
|---|---|---|---|---|
| Number of Participants Analyzed | 65 | | 76 | |
| 1 = worsen by 1 | 0 | (0%) | 0 | (0%) |
| 0 = no change | 6 | (9.23%) | 5 | (6.58%) |
| −1 = improve by 1 | 15 | (23.08%) | 18 | (23.68%) |
| −2 = improve by 2 | 15 | (23.08%) | 18 | (23.68%) |
| −3 = improve by 3 | 26 | (40%) | 33 | (43.42%) |
| −4 = improve by 4 | 3 | (4.62%) | 2 | (2.63%) |

Subjects received Placebo injections up to week 4, then at Week 16, all the subjects from Placebo arm were switched to receive Tildrakizumab up to week 44.

TABLE 38
Mean Change in Psoriasis Scalp Severity
Index Score from Baseline at Week 52

| | Placebo | Tildrakizumab |
|---|---|---|
| Number of Participants Analyzed | 114 | 117 |
| Mean Change in Psoriasis Scalp Severity Index | −30.4 | −31.1 |
| Score from Baseline at Week 52. | (15.39) | (13.96) |

Subjects will receive Placebo injections up to week 4. At Week 16, all the subjects from this arm were switched to receive Tildrakizumab up to week 44.

TABLE 39
Change From Baseline in Investigator Global
Assessment (Scalp Only) at Week 52

| | Placebo | | Tildrakizumab | |
|---|---|---|---|---|
| Number of Participants Analyzed | 97 | | 100 | |
| 1 = worsen by 1 | 1 | (1.03%) | 0 | (0%) |
| 0 = no change | 4 | (4.12%) | 3 | (38) |
| −1 = improve by 1 | 8 | (8.25%) | 8 | (8%) |
| −2 = improve by 2 | 18 | (18.56%) | 14 | (14%) |
| −3 = improve by 3 | 58 | (59.79%) | 66 | (66%) |
| −4 = improve by 4 | 8 | (8.25%) | 9 | (9%) |

Subjects received Placebo injections up to week 4, then at Week 16, all the subjects from Placebo arm were switched to receive Tildrakizumab up to week 44.

TABLE 40
Change From Baseline in Scalp Itch Numeric
Rating Scale (NRS) Score at Week 52

| | Placebo | Tildrakizumab |
|---|---|---|
| Number of Participants Analyzed | 114 | 117 |
| Change From Baseline in Scalp Itch Numeric | −5.0 | −4.7 |
| Rating Scale (NRS) Score at Week 52 | (3.04) | (3.04) |

Subjects will receive Placebo injections up to week 4. At Week 16, all the subjects from this arm were switched to receive Tildrakizumab up to week 44.

TABLE 41
Mean Change in PASI Score from Baseline at Week 52

| | Placebo | Tildrakizumab |
|---|---|---|
| Number of Participants Analyzed | 114 | 117 |
| Mean Change in PASI Score from | −16.43 | −16.84 |
| Baseline at Week 52 | (7.901) | (6.072) |

Subjects will receive Placebo injections up to week 4. At Week 16, all the subjects from this arm were switched to receive Tildrakizumab up to week 44.

TABLE 42
Change in Physician Global Assessment for
Skin (Whole Body) From Baseline at Week 52

| | Placebo | | Tildrakizumab | |
|---|---|---|---|---|
| Number of Participants Analyzed | 96 | | 100 | |
| 1 = worsen by 1 | 1 | (1.04%) | 6 | (6%) |
| 0 = no change | 8 | (8.33%) | 12 | (12%) |
| −1 = improve by 1 | 15 | (15.62%) | 12 | (12%) |
| −2 = improve by 2 | 26 | (27.08%) | 27 | (27%) |
| −3 = improve by 3 | 38 | (39.58%) | 49 | (49%) |
| −4 = improve by 4 | 8 | (8.33%) | 6 | (6%) |

Subjects received Placebo injections up to week 4, then at Week 16, all the subjects from Placebo arm were switched to receive Tildrakizumab up to week 44.

TABLE 43
Change from Baseline in Dermatology Life Quality
Index Score (Total and 6 Domain Scores) at
Measured Time Points through Week 52

| | Placebo | Tildrakizumab |
|---|---|---|
| Number of Participants Analyzed | 114 | 117 |
| Total | −12.5 (6.74) | −11.5 (7.51) |
| Symptoms and Feelings | −3.4 (1.27) | −3.2 (1.61) |
| Daily Activities | −2.8 (1.62) | −2.6 (1.90) |
| Leisure | −2.4 (1.93) | −2.2 (2.03) |
| Work and School | −0.9 (1.09) | −0.9 (1.17) |
| Personal Relationships | −1.8 (1.86) | −1.7 (1.97) |
| Treatment | −1.1 (1.26) | −0.9 (1.12) |

Subjects received Placebo injections up to week 4, then at Week 16, all the subjects from Placebo arm were switched to receive Tildrakizumab up to week 44.

TABLE 44
Reported Adverse Events through Week 72

| | Placebo | | Tildrakizumab | |
|---|---|---|---|---|
| | Affected/ | | Affected/ | |
| | At Risk (%) | | At Risk (%) | |
| Total All-Cause mortality | 0/114 | (0%) | 0/117 | (0%) |
| Serious Adverse Events | | | | |
| Total | 4/114 | (3.51%) | 3/117 | (2.56%) |
| Cardiac disorders | | | | |
| Acute myocardial infarction* | 1/114 | (0.88%) | 1/117 | (0.85%) |
| Atrioventricular block | 0/114 | (0%) | 1/117 | (0.85%) |
| complete* | | | | |
| Cardiac failure congestive* | 0/114 | (0%) | 1/117 | (0.85%) |
| Myocardial infarction* | 1/114 | (0.88%) | 0/117 | (0%) |
| Injury, poisoning | | | | |
| and procedural | | | | |
| complications | | | | |
| Exposure during pregnancy* | 1/114 | (0.88%) | 0/117 | (0%) |
| Nervous system disorders | | | | |
| Sciatica* | 0/114 | (0%) | 1/117 | (0.85%) |
| Respiratory, thoracic and | | | | |
| mediastinal disorders | | | | |
| Chronic obstructive | 1/114 | (0.88%) | 0/117 | (0%) |
| pulmonary | | | | |
| disease* | | | | |
| Other Adverse Events | | | | |
| Total | 37/114 | (32.46%) | 46/117 | (39.32%) |
| Gastrointestinal | | | | |
| disorders | | | | |
| Diarrhoea* | 3/114 | (2.63%) | 2/117 | (1.71%) |
| Immune system disorders | | | | |
| Seasonal Allergy* | 1/114 | (0.88%) | 2/117 | (1.71%) |
| Infections and | | | | |
| infestations | | | | |
| Bronchitis* | 0/114 | (0%) | 2/117 | (1.71%) |
| Covid-19* | 4/114 | (3.51%) | 4/117 | (3.42%) |
| Influenza* | 3/114 | (2.63%) | 0/117 | (0%) |
| Nasopharyngitis* | 5/114 | (4.39%) | 5/117 | (4.27%) |
| Sinusitis* | 1/114 | (0.88%) | 3/117 | (2.56%) |
| Upper Respiratory Tract | 3/114 | (2.63%) | 3/117 | (2.56%) |
| Infection* | | | | |
| Urinary Tract Infection* | 1/114 | (0.88%) | 2/117 | (1.71%) |
| Viral Upper Respiratory | 1/114 | (0.88%) | 4/117 | (3.42%) |
| Tract Infection* | | | | |
| Vulvovaginal Candidiasis* | 2/114 | (1.75%) | 0/117 | (0%) |
| Injury, poisoning | | | | |
| and procedural | | | | |
| complications | | | | |
| Ligament Sprain* | 0/114 | (0%) | 3/117 | (2.56%) |
| Investigations | | | | |
| Alanine Aminotransferase | 2/114 | (1.75%) | 1/117 | (0.85%) |
| Increased* | | | | |
| Blood Triglycerides | 2/114 | (1.75%) | 0/117 | (0%) |
| Increased* | | | | |
| Sars-Cov-2 Test Positive* | 2/114 | (1.75%) | 2/117 | (1.71%) |
| Weight Increased* | 0/114 | (0%) | 2/117 | (1.71%) |
| Metabolism and | | | | |
| nutrition disorders | | | | |
| Gout* | 0/114 | (0%) | 2/117 | (1.71%) |
| Hypercholesterolaemia* | 2/114 | (1.75%) | 0/117 | (0%) |
| Hyperlipidaemia* | 1/114 | (0.88%) | 2/117 | (1.71%) |
| Musculoskeletal and | | | | |
| connective tissue | | | | |
| disorders | | | | |
| Arthralgia* | 1/114 | (0.88%) | 2/117 | (1.71%) |
| Rheumatoid Arthritis* | 0/114 | (0%) | 2/117 | (1.71%) |
| Tendonitis* | 2/114 | (1.75%) | 0/117 | (0%) |
| Neoplasms benign, | | | | |
| malignant and unspecified | | | | |
| (incl cysts and polyps) | | | | |
| Basal Cell Carcinoma* | 0/114 | (0%) | 2/117 | (1.71%) |
| Nervous system | | | | |
| disorders | | | | |
| Headache* | 6/114 | (5.26%) | 3/117 | (2.56%) |
| Sciatica* | 0/114 | (0%) | 2/117 | (1.71%) |
| Renal and urinary | | | | |
| disorders | | | | |
| Acute Kidney Injury* | 2/114 | (1.75%) | 1/117 | (0.85%) |
| Respiratory, thoracic | | | | |
| and mediastinal | | | | |
| disorders | | | | |
| Cough* | 3/114 | (2.63%) | 0/117 | (0%) |
| Skin and subcutaneous | | | | |
| tissue disorders | | | | |
| Pruritus* | 3/114 | (2.63%) | 1/117 | (0.85%) |
| Seborrhoeic Dermatitis* | 0/114 | (0%) | 2/117 | (1.71%) |
| Urticaria* | 0/114 | (0%) | 2/117 | (1.71%) |
| Vascular disorders | | | | |
| Hypertension* | 3/114 | (2.63%) | 6/117 | (5.13%) |

Subjects received Placebo injections up to week 4, then at Week 16, all the subjects from Placebo arm were switched to receive Tildrakizumab up to week 44.
*Indicates events were collected by systematic assessment

What is claimed is:

1. A method of treating psoriasis of the scalp comprising administering an anti-IL-23p19 antibody hum13B8-b to a patient, wherein hum13B8-b comprises:
(i) a light chain polypeptide comprising the amino acid sequence of SEQ ID NO: 1, and
(ii) a heavy chain polypeptide comprising the amino acid sequence of SEQ ID NO: 2.

2. The method according to claim 1, wherein the psoriasis of the scalp is moderate to severe psoriasis of the scalp.

3. The method according to claim 1, wherein a therapeutically effective amount of hum13B8-b is administered to the patient.

4. The method according to claim 1, wherein a first dose of hum13B8-b is administered to the patient on week 0, a second dose of hum13B8-b is administered to the patient at 4 weeks, and a subsequent dose of hum13B8-b is administered to the patient every 4 to 12 weeks thereafter.

5. The method according to claim 4, wherein the first dose, the second dose, and the subsequent dose are the same.

6. The method according to claim 5, wherein the first dose, the second dose, and the subsequent dose are 100 mg.

7. The method according to claim 4, wherein the subsequent dose is administered every 12 weeks at least up to 16 weeks.

8. The method according to claim 4, wherein the subsequent dose is administered every 4 weeks at least up to 28 weeks.

9. The method according to claim 4, wherein the subsequent dose is administered every 12 weeks at least up to 40 weeks, at least up to 52 weeks, at least up to 72 weeks, or longer.

10. The method according to claim 1, wherein the antibody is administered to the patient by subcutaneous injection.

11. The method according to claim 1, wherein the patient has an affected Scalp Surface Area (SSA) of at least 50%, a Psoriasis Area and Severity Index (PASI) score of at least 16.7, and/or a Physician Global Assessment (PGA) score of 3 (“moderate”) or 4 (“severe”).

12. The method according to claim 1, wherein the patient is male, White, and/or at least 44 years of age.

13. The method according to claim 1, wherein administration of the antibody results in the patient achieving an Investigator Global Assessment (IGA) score for the scalp of “clear” or “almost clear” with at least a 2-point reduction from Baseline at Week 16.

14. The method according to claim 1, wherein administration of the antibody results in the patient achieving an Investigator Global Assessment (IGA) score for the scalp of “clear” or “almost clear” with at least a 2-point reduction from Baseline at Week 12.

15. The method according to claim 1, wherein administration of the antibody results in the patient achieving an Investigator Global Assessment (IGA) Scalp Response Rate of 0 (clear) or 1 (almost clear) at Week 12.

16. The method according to claim 1, wherein administration of the antibody results in the patient achieving Psoriasis Scalp Severity Index (PSSI) 90 at Week 16.

17. The method according to claim 1, wherein administration of the antibody results in the patient achieving Psoriasis Scalp Severity Index (PSSI) 90 at Week 12.

18. A method of treating plaque psoriasis of the scalp comprising administering a therapeutically effective amount of an anti-IL-23p19 antibody hum13B8-b to a patient in need thereof, wherein a first dose of hum13B8-b is subcutaneously administered to the patient on week 0, a second dose of hum13B8-b is subcutaneously administered to the patient at 4 weeks, and a subsequent dose of hum13B8-b is subcutaneously administered to the patient every 4 to 12 weeks thereafter; and
wherein hum13B8-b comprises:
a light chain polypeptide comprising the amino acid sequence of SEQ ID NO: 1; and
(ii) a heavy chain polypeptide comprising the amino acid sequence of SEQ ID NO: 2.

19. The method according to claim 18, wherein the first dose, the second dose, and the subsequent dose are the same.

20. The method according to claim 19, wherein the first dose, the second dose, and the subsequent dose are 100 mg.

21. The method according to claim 18, wherein the subsequent dose is administered to the patient every 12 weeks at least up to 16 weeks.

22. The method according to claim 18, wherein the subsequent dose is administered to the patient every 4 weeks at least up to 28 weeks.

23. The method according to claim 18, wherein the subsequent dose is administered to the patient every 12 weeks at least up to 40 weeks, at least up to 52 weeks, at least up to 72 weeks, or longer.

24. The method according to claim 18, wherein administration of hum13B8-b results in an Investigator Global Assessment (IGA) mod 2011 rating scale (Scalp) score of “clear” or “almost clear” with at least a 2-point reduction from the patient's baseline score.

25. The method according to claim 24, wherein administration of hum13B8-b further results in a significant improvement from the patient's baseline score for at least two of four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

26. The method according to claim 18, wherein administration of hum13B8-b results in a significant improvement from the patient's baseline score in an Investigator Global Assessment (IGA) mod 2011 rating scale (Scalp) score.

27. The method according to claim 26, wherein administration of humB13B-b further results in a significant improvement from the patient's baseline score for at least two of four parameters selected from the group consisting of: (i) Investigator Global Assessment (Scalp only) (IGA) score, (ii) Scalp Itch Numeric Rating Scale (NRS) score, (iii) Scalp Surface Area (SSA) score, and (iv) Psoriasis Scalp Severity Index (PSSI) score.

28. The method according to claim 26, wherein administration of hum13B8-b further results in a significant improvement from the patient's baseline score for the Psoriasis Scalp Severity Index (PSSI).

29. A pharmaceutical composition of an anti-IL-23p19 antibody hum13B8-b for the treatment of plaque psoriasis of the scalp in a patient, wherein hum13B8-b comprises:
(i) a light chain polypeptide comprising the amino acid sequence of SEQ ID NO: 1, and
(ii) a heavy chain polypeptide comprising the amino acid sequence of SEQ ID NO: 2; and
wherein a first dose of the pharmaceutical composition is subcutaneously administered to the patient on week 0, a second dose of the pharmaceutical composition is subcutaneously administered to the patient at 4 weeks, and a subsequent dose of the pharmaceutical composition is subcutaneously administered to the patient every 4 to 12 weeks thereafter.
